SEQUENCE LISTING (29 sequences)

SEQ ID NO: 1 - DNA, 12 bp, Artificial Sequence
  Description of Artificial Sequence Synthetic oligonucleotide
aaaaaaaaaaaa

SEQ ID NO: 2 - DNA, 24 bp, Artificial Sequence
  Description of Artificial Sequence Synthetic oligonucleotide
aaaaaaaaaaaaaaaaaaaaaaaa

SEQ ID NO: 3 - DNA, 10 bp, Artificial Sequence
  Description of Artificial Sequence Synthetic oligonucleotide
catgctactg

SEQ ID NO: 4 - DNA, 28 bp, Artificial Sequence
  Description of Artificial Sequence Synthetic oligonucleotide
tatgtctgactcactataggaagagatg

SEQ ID NO: 5 - DNA, 41 bp, Artificial Sequence
  Description of Artificial Sequence Synthetic oligonucleotide
catctcttctccgagccggtcgaaatagtgagtcagacata

SEQ ID NO: 6 - DNA, 10 bp, Artificial Sequence
  Description of Artificial Sequence Synthetic oligonucleotide
ggaagagatg

SEQ ID NO: 7 - DNA, 18 bp, Artificial Sequence
  Description of Artificial Sequence Synthetic oligonucleotide
tatgtctgactcactata

SEQ ID NO: 8 - DNA, 13 bp, Artificial Sequence
  Description of Artificial Sequence Synthetic oligonucleotide
cggatagtgttcc

SEQ ID NO: 9 - DNA, 14 bp, Artificial Sequence
  Description of Artificial Sequence Synthetic oligonucleotide
gtattagaggattc

SEQ ID NO: 10 - DNA, 53 bp, Artificial Sequence
  Description of Artificial Sequence Synthetic oligonucleotide
gaatcctctaatacactcactataggaagagatggacgtgggaacactatccg

SEQ ID NO: 11 - DNA, 51 bp, Artificial Sequence
  Description of Artificial Sequence Synthetic oligonucleotide
cacgtccatctctgcagtcgggtagttaaccgaccttcagacatagtgagt

SEQ ID NO: 12 - DNA, 24 bp, Artificial Sequence
  Description of Artificial Sequence Synthetic oligonucleotide
gaatcctctaatacactcactata

SEQ ID NO: 13 - DNA, 29 bp, Artificial Sequence
  Description of Artificial Sequence Synthetic oligonucleotide
ggaagagatggacgtgggaacactatccg

SEQ ID NO: 14 - DNA, 20 bp, Artificial Sequence
  Description of Artificial Sequence Synthetic oligonucleotide
gtgttcccacgtccatctct

SEQ ID NO: 15 - DNA, 22 bp, Artificial Sequence
  Description of Artificial Sequence Synthetic oligonucleotide
tagtgttcccacgtccatctct

SEQ ID NO: 16 - DNA, 24 bp, Artificial Sequence
  Description of Artificial Sequence Synthetic oligonucleotide
gatagtgttcccacgtccatctct

SEQ ID NO: 17 - DNA, 26 bp, Artificial Sequence
  Description of Artificial Sequence Synthetic oligonucleotide
cggatagtgttcccacgtccatctct

SEQ ID NO: 18 - DNA, 17 bp, Artificial Sequence
  Description of Artificial Sequence Synthetic oligonucleotide
atagtgagtgtattaga

SEQ ID NO: 19 - DNA, 19 bp, Artificial Sequence
  Description of Artificial Sequence Synthetic oligonucleotide
atagtgagtgtattagagg

SEQ ID NO: 20 - DNA, 21 bp, Artificial Sequence
  Description of Artificial Sequence Synthetic oligonucleotide
atagtgagtgtattagaggat

SEQ ID NO: 21 - DNA, 23 bp, Artificial Sequence
  Description of Artificial Sequence Synthetic oligonucleotide
atagtgagtgtattagaggattc

SEQ ID NO: 22 - DNA, 53 bp, Artificial Sequence; identical to SEQ ID NO: 10
  Description of Artificial Sequence Synthetic oligonucleotide

SEQ ID NO: 23 - DNA, 122 bp, Artificial Sequence
  Description of Artificial Sequence Synthetic polynucleotide
aaaaaaaaaaaaaaaaaaaaaacggatagtgttcccacgtccatctctgcagtcgggtagttaaccgaccttcagacatagtgagtgtattagaggattcaaaaaaaaaaaaaaaaaaaaaa

SEQ ID NO: 24 - DNA, 25 bp, Artificial Sequence
  Description of Artificial Sequence Synthetic oligonucleotide
ccttgtgataggcaaaaaaaaaaaa

SEQ ID NO: 25 - DNA, 26 bp, Artificial Sequence
  Description of Artificial Sequence Synthetic oligonucleotide
aaaaaaaaaaaacttaggagattatg

SEQ ID NO: 26 - DNA, 26 bp, Artificial Sequence
  Description of Artificial Sequence Synthetic oligonucleotide
actcactataggaagagatggacgtg

SEQ ID NO: 27 - DNA, 10 bp, Artificial Sequence
  Description of Artificial Sequence Synthetic oligonucleotide
actcactata

SEQ ID NO: 28 - DNA, 16 bp, Artificial Sequence
  Description of Artificial Sequence Synthetic oligonucleotide
ggaagagatggacgtg

SEQ ID NO: 29 - DNA, 36 bp, Artificial Sequence
  Description of Artificial Sequence Synthetic oligonucleotide
aaaaaaaaaaaaaaaaaaaaaaaaaaaaaaaaaaaa

(19) United States
(12) Patent Application Publication
(10) Pub. No.: US 2010/0105039 A1
(43) Pub. Date: Apr. 29, 2010

(54) LABEL-FREE COLORIMETRIC DETECTION

(76) Inventors: Yi Lu, Champaign, IL (US); Zidong Wang, Urbana, IL (US); Jung Heon Lee, Evanston, IL (US); Juewen Liu, Albuquerque, NM (US)

Correspondence Address: EVAN LAW GROUP LLC, 600 WEST JACKSON BLVD., SUITE 625, CHICAGO, IL, 60661, US

(21) Appl. No.: 12/476,756

(22) Filed: Jun. 2, 2009

Related U.S. Application Data
(60) Provisional application No. 61/058,483, filed on Jun. 3, 2008.

(51) Int. Cl. C12Q 1/68

(52) U.S. Cl. 435/6

(57) ABSTRACT

The present invention provides a sensor system kit for detecting an analyte, consisting essentially of: a nucleic acid enzyme, wherein the nucleic acid enzyme cleaves a substrate in the presence of the analyte; the substrate for the nucleic acid enzyme, comprising a polynucleotide; an aggregator; and particles.

### CROSS REFERENCE TO RELATED APPLICATION

This application claims priority to provisional application No. 61/058,483 entitled “Label-Free Colorimetric Detection” filed 3 Jun. 2008, attorney docket no. ILL10-129-PRO, the entire contents of which are hereby incorporated by reference, except where inconsistent with the present application.

### FEDERALLY SPONSORED RESEARCH OR DEVELOPMENT

The subject matter of this application may have been funded in part by the National Science Foundation (DMI-0328162 and DMR-0117792), the Department of Defense (DAAD19-03-1-0227), the Office of Science (BER), the U.S. Department of Energy (DEFG02-01-ER63179) and by the National Institute of Health (Small Business Innovation Research (SBIR) Phase I grant, Grant No. ES014125). The federal government may have certain rights in this invention.

### BACKGROUND

Besides proteins, nucleic acids have also been found to have catalytic activities in recent years. The catalytically active nucleic acids are referred to as catalytic DNA/RNA, and may also be known as DNAzymes/RNAzymes, deoxyribozymes/ribozymes, and DNA enzymes/RNA enzymes. The catalytic activity of nucleic acid-based enzymes always depends on the presence of certain cofactors, for example, metal ions. Therefore, nucleic acid enzyme-based biosensors for these cofactors (e.g. biosensors for metal ions) can be designed based on the activity of the corresponding nucleic acid enzymes.

Furthermore, a class of nucleic acids, known as aptamers, may be selected which bind to a wide range of analytes with high affinity and specificity. Aptamers are nucleic acids (such as DNA or RNA) that recognize targets with high affinity and specificity (Ellington and Szostak 1990, Jayasena 1999). Aptamers for a given target can be obtained by no more than routine experimentation. For instance, in vitro selection methods can be used to obtain aptamers for a wide range of target molecules with exceptionally high affinity, having dissociation constants as high as in the picomolar range (Brody and Gold 2000, Jayasena 1999, Wilson and Szostak 1999). For example, aptamers have been developed to recognize metal ions such as Zn(II) (Ciesiolka et al. 1995) and Ni(II) (Hofmann et al. 1997); nucleotides such as adenosine triphosphate (ATP) (Huizenga and Szostak 1995); and guanine (Kiga et al. 1998); co-factors such as NAD (Kiga et al. 1998) and flavin (Lauhon and Szostak 1995); antibiotics such as viomycin (Wallis et al. 1997) and streptomycin (Wallace and Schroeder 1998); proteins such as HIV reverse transcriptase (Chaloin et al. 2002) and hepatitis C virus RNA-dependent RNA polymerase (Biroccio et al. 2002); toxins such as cholera whole toxin and staphylococcal enterotoxin B (Bruno and Kiel 2002); and bacterial spores such as the anthrax (Bruno and Kiel 1999). Compared to antibodies, DNA/RNA based aptamers are easier to obtain and less expensive to produce because they are obtained in vitro in short time periods (days vs. months) and at limited cost. In addition, DNA/RNA aptamers can be denatured and renatured many times without losing their biorecognition ability. These unique properties make aptamers an ideal platform for designing highly sensitive and selective biosensors (Hesselberth et al. 2000).

Aptazymes (also called allosteric DNA/RNAzymes or allosteric (deoxy)ribozymes) are DNA/RNAzymes regulated by an effector (the target molecule). They typically contain an aptamer domain that recognizes an effector, and a catalytic domain (Hesselberth et al. 2000, Soukup and Breaker 2000, Tang and Breaker 1997). The effector can either decrease or increase the catalytic activity of the aptazyme through specific interactions between the aptamer domain and the catalytic domain. Therefore, the activity of the aptazyme can be used to monitor the presence and quantity of the effector. This strategy has been used to select and design aptazyme sensors for diagnostic and sensing purposes (Breaker 2002, Robertson and Ellington 1999, Seetharaman et al. 2001). In addition, general strategies to design DNA aptazymes, by introducing aptamer motifs close to the catalytic core of DNAzymes, are available (Wang et al. 2002). High cleavage activity requires the presence of effector molecules that upon binding to the aptamer motif, can allosterically modulate the activity of the catalytic core part of the aptazyme.

To assay nucleic acid enzyme activity, metallic particles can be used as detectable labels. In sensors based on aptamers using metallic particles for color detection, the cleavage of a nucleic acid substrate by the aptazyme (upon binding of an effector) may be detected by color changes. An example of such a sensor is a nucleic acid enzyme directed disassembly sensor.

Typically, a nucleic acid enzyme directed disassembly sensor has three parts:

(1) a nucleic acid enzyme and a co-factor, such as a metal ion that catalyzes substrate cleavage;

(2) a nucleic acid substrate for the nucleic acid enzyme, wherein interior portions of the substrate sequence are complementary to portions of the enzyme sequence; and

(3) particles attached to polynucleotides that are complementary to the 3′- and 5′-termini of the substrate.

To detect the target cofactor or effector, the complementary portions of the polynucleotides are annealed in the presence of a sample suspected of containing the targeted cofactor or effector. If the cofactor or effector is absent, the nucleic acid enzyme is either inactive or shows substantially reduced activity, resulting in no or little substrate cleavage and thus aggregation of the particles. If the cofactor or effector is present, the enzyme becomes active and cleaves the substrate, preventing aggregate formation because the link between the particles is broken by enzymatic cleavage.

In the case of gold nanoparticles (AuNPs), the aggregated state displays a blue color, while the dispersed state (or the non-aggregate state) is red in color. The presence of the target analyte as a cofactor or effector can be detected based on the appearance of the color of the sensor system.

Since the degree of cleavage is reflected in the degree of color change, the target cofactor or effector concentration can be quantified. For example, simple spectrometry may be used for sensitive detection. Not only can color change be used for detection and quantifying, other results of the cleavage may be employed, such as precipitation. By replacing the aptamer domain of the aptazyme with the sequence of an aptamer recognizing a different pre-selected effector, colorimetric sensors for any desired effector can be easily made and used.

These colorimetric biosensors based on the nucleic acid enzyme directed disassembly, or assembly, of nanoparticles have been designed, for example, to detect Pb(II) and adenosine (see, for example, U.S. Pat. No. 6,706,474; U.S. Pat. Publ. Nos. 2003/0215810, 2004/0175693, 2006/0166222; U.S. patent application Ser. No. 10/756,825). However, this type of sensor has a detection limit of 100 nM, which is higher than the maximum contamination level (MCL) of 72 nM for lead in drinking water as defined by the U.S. Environmental Protection Agency (EPA). Such a high detection limit could be due to the need to cleave a number of substrates, which link the particles together, before the color change can occur. Furthermore, such a sensor system requires a relatively long time and significant effort to prepare before use.

Rothberg and co-workers reported that single stranded DNA (ssDNA) and double stranded DNA (dsDNA) have different absorption properties on AuNP. Since ssDNA is flexible and can partially uncoil its bases, it can be easily absorbed on AuNPs and thus prevent salt induced AuNP aggregation, by enhancing the electrostatic repulsion between ssDNA-absorbed AuNPs. dsDNA, in contrast is stiffer and has an exposed negatively charged backbone, and the strong repulsion between dsDNA and negatively charged AuNPs results in negligible binding, which cannot prevent salt-induced AuNP aggregation. Based on this phenomenon, hybridization assays to detect specific DNA or RNA sequences using unmodified AuNPs have been developed. Since no chemical modification is necessary for either the DNA/RNA strands or the AuNPs, the sensors are also called label-free colorimetric detection sensors. In addition, aptamer-based label-free colorimetric sensors that rely on the binding of targets to aptamers have also been reported.

### SUMMARY

In a first aspect, the present invention provides a sensor system kit for detecting an analyte, consisting essentially of: a nucleic acid enzyme, wherein the nucleic acid enzyme cleaves a substrate in the presence of the analyte; the substrate for the nucleic acid enzyme, comprising a polynucleotide; an aggregator; and particles.

In a second aspect, the present invention provides a sensor system kit for detecting an analyte, comprising: a nucleic acid enzyme, wherein the nucleic acid enzyme cleaves a substrate in the presence of the analyte; the substrate for the nucleic acid enzyme, comprising a polynucleotide; an aggregator; and particles, wherein the particles are not attached to oligonucleotides that are hybridized to the substrate.

In a third aspect, the present invention provides a method of detecting an analyte, comprising: mixing a sample with a sensor; to form a product; and mixing the product with an indicator, wherein the sensor comprises: a nucleic acid enzyme, wherein the nucleic acid enzyme cleaves a substrate in the presence of the analyte; the substrate for the nucleic acid enzyme, comprising a polynucleotide; and an aggregator, and the indicator comprises particles, wherein the particles aggregate in the presence of the aggregator unless in the presence of sufficient ssDNA.

### BRIEF DESCRIPTION OF THE DRAWINGS

FIG. 1 illustrates a label-free colorimetric sensor for Pb²⁺.

FIG. 2 illustrates the quenching effect of Pb²⁺-induced cleavage reaction by the addition of EDTA.

FIG. 3A illustrates the calibration curve of a label-free colorimetric sensor for Pb²⁺. FIG. 3B shows the color change of a AuNP solution with different concentrations of Pb²⁺ in a solution at a pH of 7.2. FIG. 3C shows the color change of a AuNP solution with 1 μM of various metal ions including Pb²⁺, in a solution at a pH of 7.2.

FIG. 4A illustrates the calibration curve of a label-free colorimetric sensor at a pH of 5.5. FIG. 4B shows the color change of a AuNP solution at different concentrations of Pb²⁺ in a solution at a pH of 5.5.

FIG. 5 illustrates the scheme of a nucleic acid enzyme directed disassembly sensor for uranyl. FIG. 5A shows the complex of the sensor in the absence of uranyl. FIG. 5B illustrates the complex in the presence of uranyl. FIG. 5C illustrates the disassembly of an aggregate of the sensor.

FIG. 6A illustrates the melting curve of aggregates with and without uranyl. FIG. 6B illustrates an assay of the cleavage kinetics in the presence of uranyl. FIG. 6C shows the design of a nucleic acid enzyme directed disassembly sensor for uranyl including the sequences of invasive DNAs and Arm strands.

FIG. 7A shows the background increase of aggregates with invasive DNA strands in the absence of uranyl. FIG. 7B shows the differences in disassembly kinetics between different aggregates. FIGS. 7C and 7D show a comparison of the disassembly of different aggregates with and without invasive DNA. FIGS. 7E and 7F illustrate the effect of longer invasive DNAs in aggregates.

FIG. 8A illustrates the kinetics of the disassembly of AuNP aggregates at various uranyl concentrations. FIG. 8B is a calibration curve of a disassembly sensor for uranyl. FIG. 8C shows the disassembly of AuNPs in the presence of various metal ions including uranyl. FIG. 8D shows the color change of AuNP aggregates in the presence of different metal ions.

FIG. 9 illustrates a label-free colorimetric sensor for uranyl.

FIG. 10A illustrates the dependence of the extinction ratio on the ssDNA/AuNPs ratio. FIG. 10B shows the color change of AuNPs at different ssDNA/AuNP ratios.

FIG. 11A illustrates the quenching effect on a label-free uranyl sensor. FIG. 11B shows the color change difference of the sensor without or without Tris base solution.

FIG. 12A is a calibration curve of a label-free uranyl sensor. FIG. 12B shows the color change of a AuNP solution at different concentrations of uranyl. FIG. 12C shows the color change of a AuNP solution with various metal ions, including uranyl.

### DEFINITIONS

A “co-factor” is an ion or molecule involved in the catalytic process of nucleic acid enzyme-catalyzed reactions and is required for catalytic activity.

“Aptamer” refers a polynucleotide which contains an effector binding site. An “effector binding site” may be “specific,” that is, binding only one effector molecule in the presence of other effector molecules. An “effector” is a molecule that, when bound to an enzyme having an effector binding site, can enhance or inhibit enzyme catalysis. An “effector binding site” may be “specific,” that is, binding only one effector molecule in the presence of other effector molecules. An example of effector binding site specificity is when only an adenosine molecule binds in the presence of many other similar molecules, such as cytidine, gaunosine and uridine. Alternatively, an effector binding site may be “partially” specific (binding only a class of molecules), or “non-specific” (having molecular promiscuity). Examples of effectors include environmental pollutants, such as nitrogen fertilizers, pesticides, dioxin, phenols, or 2,4-dichlorophenoxyacetic acid; heavy metal ions, such as Pb(II), Hg(II), As(III), UO₂(II), Fe(III), Zn(II), Cu(II), or Co(II); biological molecules, such as glucose, insulin, hCG-hormone, HIV or HIV proteins; chemical and biological terrorism agents, such as anthrax, small pox, or nerve gases; explosives, such as TNT or DNT; drugs, such as cocaine or antibiotics.

A “nucleic acid enzyme” is an enzyme that principally contains nucleic acids, such as ribozymes (RNAzymes), deoxyribozymes (DNAzymes), and aptazymes. Nucleic acids may be natural, unnatural or modified nucleic acids. Peptide nucleic acids (PNAs) are also included. A nucleic acid enzyme requires a “co-factor” for efficient substrate cleavage and/or specific effector binding. Common co-factors include Mg(II), Ca(II), Zn(II), Mn(II), Co(II) and Pb(II).

“Polynucleotide” refers to a nucleic acid sequence having at least two nucleotides. Polynucleotides may contain naturally-occurring nucleotides and synthetic nucleotides. PNA molecules are also embraced by this term.

“Sensitivity” refers to the smallest increase of a cofactor or effector concentration that can be detected by the sensor.

“Detection limit” refers to the limits of detection of an analytical device. In the context of the DNAzyme- and aptazyme-based sensors of the present invention, detection limit refers to the lowest concentration of a cofactor or effector that the sensor can differentiate from the background.

“Base-pairing” or “hybridization” refers to the ability of a polynucleotide to form at least one hydrogen bond with a nucleotide under low stringency conditions. The nucleotide may be part of a second polynucleotide or to a nucleotide found within the first polynucleotide. A polynucleotide is partially complementary to a second polynucleotide when the first polynucleotide is capable of forming at least one hydrogen bond with the second polynucleotide. To be partially complementary, a polynucleotide may have regions wherein base pairs may not form surrounded by those regions that do, forming loops, stem-loops, and other secondary structures.

“Aptazyme” refers to a nucleic acid enzyme that includes an aptamer region which binds an effector. The binding of the effector can enhance or inhibit catalysis.

“Ionic strength modifier” refers to a compound that changes the ionic strength of a solution. Example ionic strength modifiers include inorganic salts, organic salts, acids, bases, and buffers.

### DETAILED DESCRIPTION

In most previously reported studies, ssDNA is absorbed on AuNPs surfaces first and then the salt is subsequently added, to induce the color change. Nucleic acid enzyme-based systems, however, contain a large mismatch between the enzyme strand and the substrate strand, which causes dehybridization of the complex within seconds in the absence of salt. For example, 24-mer dsDNA has been reported to remain hybridized for about 10 minutes in an Au colloid solution without salt, while introduction of a single mismatch will decrease the stability of the dsDNA and cause dehybridization in 5 minutes.

Therefore, unlike previously reported studies, in order to combine a nucleic acid enzyme-based system and salt-induced particle aggregation, the DNA solution added to the AuNP solution must have a sufficiently high ionic strength to keep the complex hybridized. In this case, since the stability of the AuNPs is determined by the competition between the ssDNA absorption on the AuNPs and electrostatic screening caused by salts introduced to the AuNP solution at the same time, it was unknown whether DNA can still be absorbed on AuNPs effectively and prevent aggregation in the presence of salts.

The present invention provides a label-free nucleic acid enzyme-based sensor using unmodified nanoparticles. Similarly to previous nucleic acid enzyme-based sensors, the sensor of the invention features a nucleic acid enzyme and a substrate for the nucleic acid enzyme. The nanoparticles, however, are unmodified, and are not required to be linked to polynucleotides that hybridize to the substrate of the nucleic acid enzyme.

The invention makes use of the discovery that unmodified nanoparticles can still be stabilized against aggregation by ssDNA in a solution of sufficient ionic strength to otherwise induce aggregation of the particles. Moreover, it has also been discovered that such a sensor featuring unmodified nanoparticles has a detection limit lower than previous sensors based on nanoparticles covalently attached to polynucleotides.

This label-free sensor is easy to use and the sensing process can be carried out in less than ten minutes. In addition, the sensing process can be quenched for reproducible and quantitative sensing. Furthermore, the dynamic range of the sensor for the same analyte can be tuned, allowing for sensors for different applications.

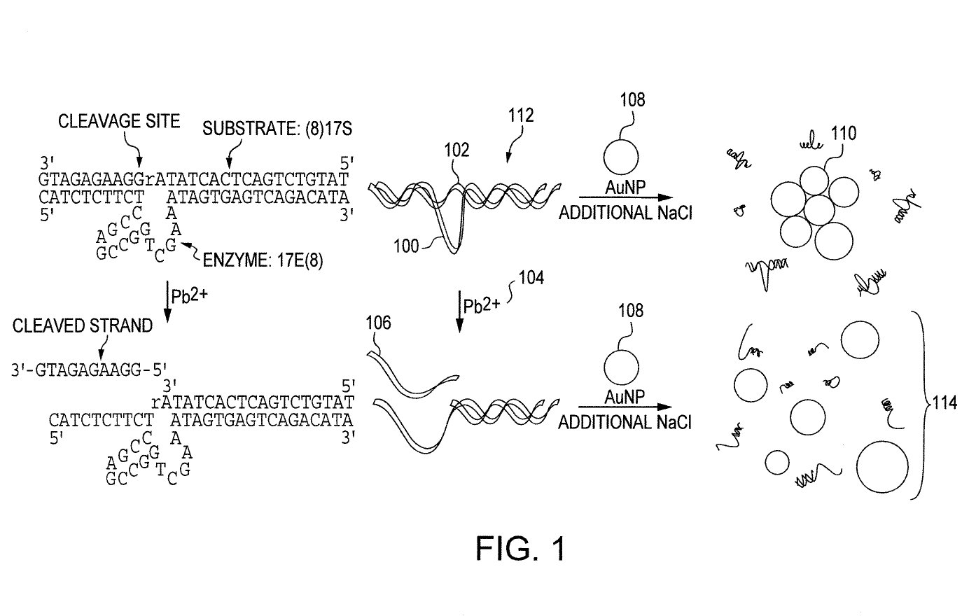

As illustrated in FIG. 1, to detect the target analyte, for example Pb²⁺, a sample suspected of containing the target analyte 104 is mixed with a sensor that includes the nucleic acid enzyme 100 and substrate 102 together in a solution of appropriate ionic strength. The enzyme and substrate are hybridized to form complex 112. If the analyte is absent, the nucleic acid enzyme is either inactive or shows little activity, resulting in no or little substrate cleavage. If the analyte is present, the enzyme becomes active and cleaves the substrate, yielding single-stranded cleavage product 106. An aggregator, for example salt, may be added to the solution, and an indicator including nanoparticles 108, for example AuNP, is then added. The cleavage product is absorbed on the nanoparticles, preventing aggregation of the nanoparticles because of enhanced electrostatic repulsion between the nanoparticles, and thereby yielding unaggregated particles 114. If the analyte is not present, the enzyme does not become active, allowing the unmodified particles to form aggregate 110.

The enzyme is a nucleic acid enzyme that catalyzes the cleavage of a nucleic acid in the presence of an analyte. The nucleic acid enzyme may be RNA (ribozyme), DNA (deoxyribozyme), a DNA/RNA hybrid enzyme, or a peptide nucleic acid (PNA) enzyme. PNAs comprise a polyamide backbone and nucleoside bases (available from, e.g., Biosearch, Inc. (Bedford, Mass.)). Ribozymes that may be used include group I and group II introns, the RNA component of the bacterial ribonuclease P, hammerhead, hairpin, hepatitis delta virus and Neurospora VS ribozymes. Also included are in vitro selected ribozymes, such as those previously isolated (Tang and Breaker 2000). Ribozymes tend to be less stable than deoxyribozymes; thus deoxyribozymes are preferred. Deoxyribozymes with extended chemical functionality are also desirable (Santoro et al., 2000).

A large variety of nucleic acid enzymes are known. Several such enzymes and the analytes they are responsive to are reported below in Table A:

TABLE A

| Analyte | Reference/s |
|---|---|
| Pb(II) | 1). Santoro, S. W.; Joyce, G. F. Proc. Natl. Acad. Sci. U.S.A. 1997, |
| | 94, 4262-4266. |
| | 2). Faulhammer, D.; Famulok, M. Angew. Chem., Int. Ed. Engl. |
| | 1996, 35, 2837-2841. |
| | 3). Li, J.; Zheng, W.; Kwon, A. H.; Lu, Y. Nucleic Acids Res. 2000, |
| | 28, 481-488. |
| | 4). R. P. G. Cruz, J. B. Withers, Y. Li, Chem. Biol. 2004, 11, 57. |
| Cu(II) | 1). Carmi, N.; Shultz, L. A.; Breaker, R. R. Chem. Biol. 1996, 3, |
| | 1039-1046. |
| | 2). Carmi, N.; Balkhi, H. R.; Breaker, R. R. Proc. Natl. Acad. Sci. |
| | U.S.A. 1998, 95, 2233-2237. |
| Zn(II) | Santoro, S. W.; Joyce, G. F.; Sakthivel, K.; Gramatikova, S.; Barbas, |
| | C. F., III. J. Am. Chem. Soc. 2000, 122, 2433-2439. |
| Mg(II) | Santoro, S. W.; Joyce, G. F. Proc. Natl. Acad. Sci. U.S.A. 1997, 94, |
| | 4262-4266. |
| Mn(II) | Liu, Z.; Mei, S. H. J.; Brennan, J. D.; Li, Y. J. Am. Chem. Soc. 2003, |
| | 725, 7539-7545. |
| Mn(II) & | Liu, Z.; Mei, S. H. J.; Brennan, J. D.; Li, Y. J. Am. Chem: Soc. 2003, |
| Ni(II) | 125, 7539-7545. |
| Co(II) | Mei, S. H. J.; Liu, Z.; Brennan, J. D.; Li, Y. J. Am. Chem. Soc. 2003, |
| | 125, 412-420. |
| Co(II) | Seetharaman, S.; Zivarts, M.; Sudarsan, N.; Breaker, R. R. Nature |
| | Biotechnology 2001, 79, 336-341. |
| Co(II) | Bruesehoff, P., J.; Li, J.; Augustine, I. A. J.; Lu, Y. Combinat. Chem. |
| | High Throughput Screening, 2002, 5, 327-335. |
| Zn(II) | Bruesehoff, P., J.; Li, J.; Augustine, I. A. J.; Lu, Y. Combinat. Chem. |
| | High Throughput Screening, 2002, 5, 327-335. |
| ATP | Tang, J.; Breaker, R. R. Chem. Biol. 1997, 4, 453-459. |
| HIV-1-RT | Hartig, J. S.; Famulok, M. Angew. Chem., Int. Ed. Engl. 2002, 41, |
| | 4263-4266. |
| cGMP | Koizumi, M.; Soukup, G. A.; Kerr, J. N. Q.; Breaker, R. R. Nat. |
| | Struct. Biol. 1999, 6, 1062-1071. |
| cCMP | Koizumi, M.; Soukup, G. A.; Kerr, J. N. Q.; Breaker, R. R. Nat. |
| | Struct. Biol. 1999, 6, 1062-1071. |
| cAMP | Koizumi, M.; Soukup, G. A.; Kerr, J. N. Q.; Breaker, R. R. Nat. |
| | Struct. Biol. 1999, 6, 1062-1071. |
| FMN | Soukup, G. A.; Breaker, R. R. Proc. Natl. Acad. Sci. U.S.A. 1999, |
| | 96, 3584-3589. |
| Theo | Soukup, G. A.; Breaker, R. R. Proc. Natl. Acad. Sci. U.S.A. 1999, |
| | 96, 3584-3589. |
| Aspartame | Ferguson, A.; Boomer, R. M.; Kurz, M.; Keene, S. C.; Diener, J. L.; |
| | Keefe, A. D.; Wilson, C.; Cload, S. T. Nucleic Acids Res. 2004, 32, |
| | 1756-1766. |
| Caffeine | Ferguson, A.; Boomer, R. M.; Kurz, M.; Keene, S. C.; Diener, J. L.; |
| | Keefe, A. D.; Wilson, C.; Cload, S. T. Nucleic Acids Res. 2004, 32, |
| | 1756-1766. |

The single stranded product formed by the cleavage of the substrate contains preferably 1 to 15 nucleotides. More preferably, the single stranded product contains 5 to 12 nucleotides.

The nanoparticles may be of any material that changes in color on aggregation or disaggregation. Example materials include metals and semiconductors, or latex spheres coated with metals or semiconductors. Particularly preferred materials include silver and gold colloids.

The nanoparticles are preferably not covalently functionalized with oligonucleotides, in particular oligonucleotides that hybridize with the substrate. Nanoparticles functionalized with oligonucleotides undergo salt induced aggregation at much higher salt concentrations than unmofied particles, thereby having a detrimental effect on the functioning of the sensor. In disassembly sensors, oligonucleotide-functionalized nanoparticles aggregate by mechanisms differing from that of the present invention. In addition, disassembly sensors are inferior in sensitivity to that of the invention, as shown below.

Aggregation of the nanoparticles is induced by an aggregator. The aggregator is preferably an ionic strength modifier, for instance an inorganic salt such as NaCl, KCl, LiCl, NaBr, KBr, and LiBr. Particularly preferred is NaCl. The aggregator is preferably already present in the sensor, and may also be present in the sample suspected of containing the target analyte. More aggregator may also be added before, together with or after the addition of the indicator.

In some cases, the cleavage of the substrate can occur very quickly, and measuring the dependency of color change on analyte concentration can therefore be difficult. To address this issue, a quencher may be added to quench the cleavage reaction at a selected point. Metal ion chelators are a preferred class of quenchers. Particularly preferred is ethylenediaminetetraacetic acid (EDTA). The dynamic range of the sensor may also be tuned to fit different concentration ranges of the analyte according to the application at hand. When the kinetics of the nucleic acid enzyme are influenced by pH, they can be slowed down or accelerated by the addition of a pH modifier, such as an acid, base, or buffer, thereby shifting the dynamic range to lower or higher concentrations of the analyte as needed. Furthermore, since the cleavage reaction can be quenched by addition of quenchers, it is also possible to further tune the dynamic range simply by changing the reaction time.

Different aggregation states of the particles results in different colors. For example, a large degree of gold particle aggregation displays blue colors while a small degree of particle aggregation displays red colors. Furthermore, the amount of substrate cleavage and thus the degree of aggregation depends on the concentration of the analyte. A low analyte concentration results in only partial substrate cleavage that produces a mixture of single particles and aggregates, allowing for semi-quantitative or qualitative assays. The color difference can be amplified to improve sensitivity. For a quantitative measurement, the optical spectra of the assay mixture are determined. In addition to color change, the formation of aggregates of the particles, or precipitation of aggregated particles may also be monitored. Color changes can be observed with the naked eye or spectroscopically. The formation of aggregates can be observed by electron microscopy or by nephelometry; precipitation of aggregated particles can be observed with the naked eye or microscopically. In the case of label free detection, as the analyte-induced DNA cleavage reaction does not occur any longer after quenching, and the color of the mixture is stable at about 15 minutes after the addition of AuNP, the color change of AuNP can be observed by naked eye and compared directly to the concentration of target analytes.

To facilitate the observation of a color change, the color may be observed on a background of a contrasting color. When gold particles are used, the observation of a color change is facilitated by spotting a sample of the hybridization solution on a solid white surface (such as silica or alumina TLC plates, filter paper, cellulose nitrate membranes, and nylon membranes) and allowing the spot to dry. Initially, the spot retains the color of the hybridization solution (which ranges from pink/red, in the absence of aggregation, to purplish-red/purple, if there has been aggregation of gold particles). On drying, a blue spot develops if aggregation is present prior to spotting; a pink spot develops if dispersion occurred. The blue and the pink spots are stable and do not change on subsequent cooling or heating or over time. They provide a convenient permanent record of the test. No other steps are necessary to observe the color change.

Alternatively, assay results may be visualized by spotting a sample onto a glass fiber filter for use with gold particles. After rinsing with water, a spot comprising the aggregates is observed. Additional methods are also available for visualizing assay results (Mirkin et al. 2002).

In this embodiment, the visualization species is provided by particles that are preferably not covalently functionalized with oligonucleotides. When the analyte is present, the enzyme becomes active and cleaves the substrate, and the single-stranded cleavage product prevents the visualization species from forming an aggregate. Since different enzymes which specifically bind different analytes may be designed which will all not form an aggregate with the same visualization species, all parts of the analytical test may be the same for different analytes, as long as the analysis chemistry reagents contain an enzyme which specifically activated by the analyte of interest.

The assay may also be carried out in lateral flow devices, such as those disclosed in U.S. Published Pat. Appl. No. 20070269821. FIG. 13 represents an analysis 1300 for determining the presence of an analyte 1302 (not shown) with a lateral flow device, such as the device 1305. The lateral flow device 1305 is depicted with a reaction area 1320, and first and second visualization zones 1340, 1350, respectively. The first visualization zone 1340 is prepared with a capture species 1345, while the second visualization zone 1350 is treated with a trapping species 1355.

In one example lateral flow device, the reaction area 1320 is treated with analysis chemistry reagents that release a visualization species in the presence of the analyte 1302. To begin the analysis 1300, a sample 1301 (not shown) suspected of containing the analyte 1302 is deposited on the reaction zone 1320. A liquid eluent, such as water including an aggregator, is then applied to the left side of the device 1305. The eluent may be any liquid that does not interfere with the analysis chemistry and that has the ability to move the visualization species from the reaction zone 1320 and through the visualization zones 1340, 1350. Preferably, the eluent is an aqueous solution. As the liquid travels through the reaction zone 1320 and through the visualization zones 1340, 1350, two scenarios are possible, illustrated from the top down on the right side of FIG. 13.

Post analysis lateral flow device 1364 depicts a failed test where neither the visualization species 1332, nor the verification species 1342 reaches the visualization zones 1340, 1350. The failure of the verification species 1342 to reach the visualization zone 1350 may mean that the sample 1301 was incompatible with the analysis chemistry or that the liquid eluent failed to transport the verification species 1342. In either instance, the analysis failed.

Post analysis lateral flow device 1362 represents the scenario when the verification species 1342 is trapped by the trapping species 1355 present in the second visualization zone 1350. The device 1362 shows a color change in the second visualization zone 1350 due to the arrival of the verification species 1342. Thus, the analysis is successful, but the sample lacked the analyte required to activate the analysis chemistry.

Post analysis lateral flow device 1360 represents the scenario when the visualization species 1332 is hybridized by the capture species present in the first visualization zone 1340 and the verification species 1342 is trapped in the second visualization zone 1350. Thus, the analysis is successful and the sample included the analyte which activated the analysis chemistry to release the visualization species.

A variety of analysis chemistry, and hence analysis chemistry reagents, may be used, and may be selected based on the choice of analyte and label. In one example lateral flow device, the reagents in the reaction zone include a nucleic acid enzyme, a substrate, nanoparticles such as AuNP, optionally together with an aggregator. Alternatively, the aggregator may be present in the sample and/or the eluent. The visualization zone 1340 is treated with a capture species that binds to the nanoparticles, for example sulfur-containing compounds that bind to gold.

If the analyte is not present in the sample, the enzyme does not become active, allowing the unmodified particles to form aggregates. If the analyte is present, the enzyme becomes active and cleaves the substrate, yielding a single-stranded cleavage product. The cleavage product is absorbed on the nanoparticles, preventing aggregation of the nanoparticles and thereby yielding unaggregated particles that serve as visualization species. The eluent moves the unaggregated nanoparticles to the visualization zone, where they bind to the capture species and signal the presence of the analyte by their color.

In a second example lateral flow device, the reagents in the reaction zone include a nucleic acid enzyme and a substrate. The single-stranded cleavage product serves as the visualization species, and nanoparticles in the visualization zone serve as capture species. The aggregator may be present in the reaction zone, the visualization zone and/or the eluent. If the analyte is present, the eluent moves the single-stranded product to the visualization zone, where binding to the particles prevents the formation of aggregates. Conversely, the absence of the analyte is signaled by the formation of aggregates. The difference in color between aggregated and unaggregated nanoparticles in the visualization zone indicates the presence or absence of the analyte.

The targeted analyte can be detected in a variety of samples, including biological samples. Standards containing known amounts of the cofactor or effector may be assayed along side the unknown sample, and the color changes compared. Alternatively, standard color charts, similar to those used with pH papers, may be provided.

The invention provides sensor system kits for detecting analytes as cofactors or effectors. In one embodiment, the kit includes at least a first container and a second container. The first container contains the sensor. The second container contains the indicator.

When a kit is supplied, the different components of the composition may be packaged in separate containers and admixed immediately before use. Such packaging of the components separately permits long-term storage of the active components.

The reagents included in the kits can be supplied in containers of any sort such that the life of the different components are preserved and are not adsorbed or altered by the materials of the container. For example, sealed glass ampules may contain one of more of the reagents, or buffers that have been packaged under a neutral, non-reacting gas, such as nitrogen. Ampules may consist of any suitable material, such as glass, organic polymers, such as polycarbonate, polystyrene, etc.; ceramic, metal or any other material typically employed to hold similar reagents. Other examples of suitable containers include simple bottles that may be fabricated from similar substances as ampules; and envelopes that may comprise foil-lined interiors, such as aluminum or an alloy. Other containers include test tubes, vials, flasks; bottles, syringes, or the like. Containers may have a sterile access port, such as a bottle having a stopper that can be pierced by a hypodermic injection needle. Other containers may have two compartments that are separated by a readily removable membrane that upon removal permits the components to be mixed. Removable membranes may be glass, plastic, rubber, etc.

The kits may also contain other reagents and items useful for detecting the target analyte. The reagents may include standard solutions containing known quantities of the analyte, dilution and other buffers, pretreatment reagents, etc. Other items which may be provided as part include a backing (for visualizing aggregate break down), such as a TLC silica plate; microporous materials, syringes, pipettes, cuvettes and containers. Standard charts indicating the appearance of the particles in various aggregation states, corresponding to the presence of different amounts of the cofactor or effector being tested, may be provided.

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, etc. Detailed instructions may not be physically associated with the kit; instead, a user may be directed to an internet web site specified by the manufacturer or distributor of the kit, or supplied as electronic mail.

### EXAMPLES

### Example 1

### Colorimetric Pb²⁺ Biosensor

The design of a label-free sensor for Pb²⁺ is shown in FIG. 1. It is based on an 8-17 DNAzyme that has been shown to be highly specific for Pb²⁺. The 8-17 DNAzyme is composed of a substrate strand extended by 8 bases at the 5′ end ((8)17S) and an enzyme strand extended by 8 complimentary bases at the 3′ end (17E(8)). The 8 base pair extension allows stable hybridization between the substrate and enzyme strands at ambient temperature, while still allowing release of single stranded ssDNA at the other end upon cleavage in the presence of Pb²⁺.

Upon addition of Tris and sodium chloride (NaCl) to adjust ionic strength, followed by addition of AuNPs, the released ssDNA can be absorbed onto the AuNPs and prevent the individual red AuNPs from forming blue aggregates under high salt conditions. NaCl concentration is kept higher than 100 mM for the entire process so that non-specific dissociation of the complex can be prevented. In the absence of Pb²⁺ or in the presence of other metal ions, however, no cleavage reaction should occur, and the enzyme-substrate complex would not be able to stabilize individual red AuNPs, resulting in purple-blue AuNPs aggregates.

After adding the Au colloid, the UV-vis spectrometer was used to record the plasmon peak shift of the AuNP colloids. The ratio of extinction at 522 and 700 nm was chosen to monitor the amount of AuNP aggregation that causes the color variation. A lower ratio is associated with aggregated nanoparticles of a blue color, while a higher ratio is associated with dispersed nanoparticles of red color.

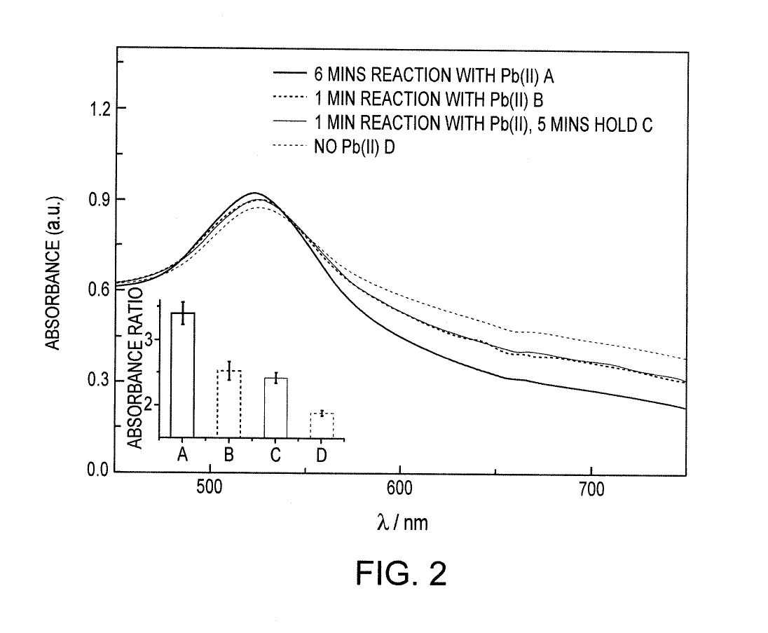

When the AuNPs were added to DNAzyme complex without Pb²⁺, an extinction ratio of about 2.0 was observed (FIG. 2), which indicates high AuNP aggregations in the present of high salt conditions (0.5 mM Tris and 100 mM NaCl). On the other hand, when the DNAzyme complex was treated with 500 nM Pb²⁺ for 6 minutes, AuNP had extinction ratio of about 3.4, which suggested much less AuNP aggregations due to cleavage and release of ssDNA product that bind to AuNP and prevent aggregation.

To quench this Pb²⁺ induced cleavage reaction with EDTA for label-free colorimetric sensing, a 6 mM EDTA solution was added to the above solution after 1 min of reaction, together with Tris and NaCl. An extinction ratio of about 2.5 was observed, indicating less AuNP aggregation than the reaction without Pb²⁺, due to the 1 min reaction time with Pb²⁺, but considerably more than in the case of the 6 min reaction with Pb²⁺. This result is attributable to EDTA induced quenching of the cleavage reaction.

In order to ensure that the EDTA quenching was complete at such a concentration and no further Pb²⁺ induced reaction occurred afterwards, a control experiment was carried out to extend the interval between EDTA quenching of the reaction and AuNP addition from a few seconds to 5 min. A minor difference in the extinction ratio was observed between the two time intervals, suggesting there was no further Pb²⁺ induced cleavage reaction. Taken together, these results indicate that the added EDTA solution could quench the reaction very effectively and timely. As Pb²⁺ induced DNA cleavage reaction does not occur any more after quenching, and the color of the mixture solution after addition of AuNP is also stable (no observable change in 15 min), the color change of AuNP can be observed by the naked eye and compared directly to the concentration of target metal ions.

Since EDTA contains charges, amines and carboxylic acids, which might interact with AuNPs, its effects on AuNP aggregation in the absence of DNA were investigated, and it was found that no AuNP aggregation was observed when up to 20 mM EDTA was added. Since 6 mM EDTA was added to the solution for a final concentration of 4 mM, its effect on AuNP aggregations and thus color changes is negligible in comparison to 100 mM NaCl added to the solution.

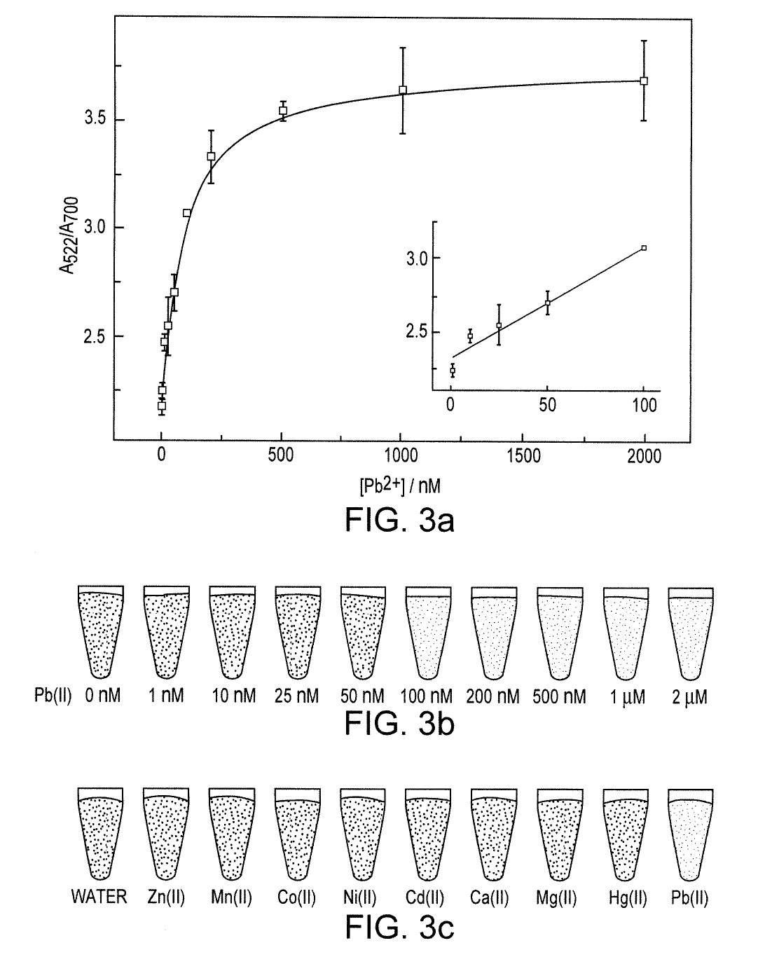

In order to determine the sensitivity of sensor, the DNAzyme complex formed in 10 mM Tris buffer pH 7.2 with 100 mM NaCl was treated with various concentrations of Pb²⁺ and the cleavage reaction was quenched by adding EDTA solution at 6 min after the addition of Pb²⁺, followed by addition of Au colloids for detection. Plasmon resonance peak shift of AuNPs was monitored by UV-vis and the extinction ratio between 522 nm and 700 nm was compared at different Pb²⁺ concentrations (FIG. 3A). The detection limit was determined to be 3 nM, which is even lower than the detection limit of fluorescent sensors for lead (10 nM) and the MCL for lead (72 nM) defined by the EPA.

The calibration curve saturated at 1 μM, which means that the dynamic range is from 3 nM to 1 μM with a linear fitting range from 3 nM to 100 nM. The color change is shown in FIG. 3b. Since the Pb²⁺ dependent cleavage reaction was made in a concentrated DNAzyme solution at a pH of 7.2 and then transferred in a AuNP solution, the cleavage reaction was very efficient. Accordingly, only a small amount of ssDNA is needed induce color change, and the method can be very sensitive. To investigate the selectivity of this sensor, several metal ions including lead were added to sensor solution separately and their color changes are shown in FIG. 3c. The result clearly shows that the sensor responded only in the presence of Pb²⁺, demonstrating its selectivity.

A tunable dynamic range is important for practical applications as the desirable concentrations for the same target analyte can be different for various applications. For example, while the maximum contamination level for lead in drinking water is 72 nM, the lead level extracted from paint is in μM range, whereas the level for lead in dusts is even more diverse depending on locations where the dusts are collected. Therefore, a sensor with a dynamic range of 3 nM to 1 μM, while excellent for detection of lead in water, would not be ideal for detecting lead in paints or dust.

In order to tune the dynamic range and fit the sensor for different detection requirements, pH was investigated as a tunable parameter. Since biochemical study of the DNAzyme suggested that the kinetics of the reaction is slower at lower pH, it was hypothesized that a higher concentration of Pb²⁺ may be needed to achieve the same extent of cleavage at a low pH in the same unit time as at a high pH.

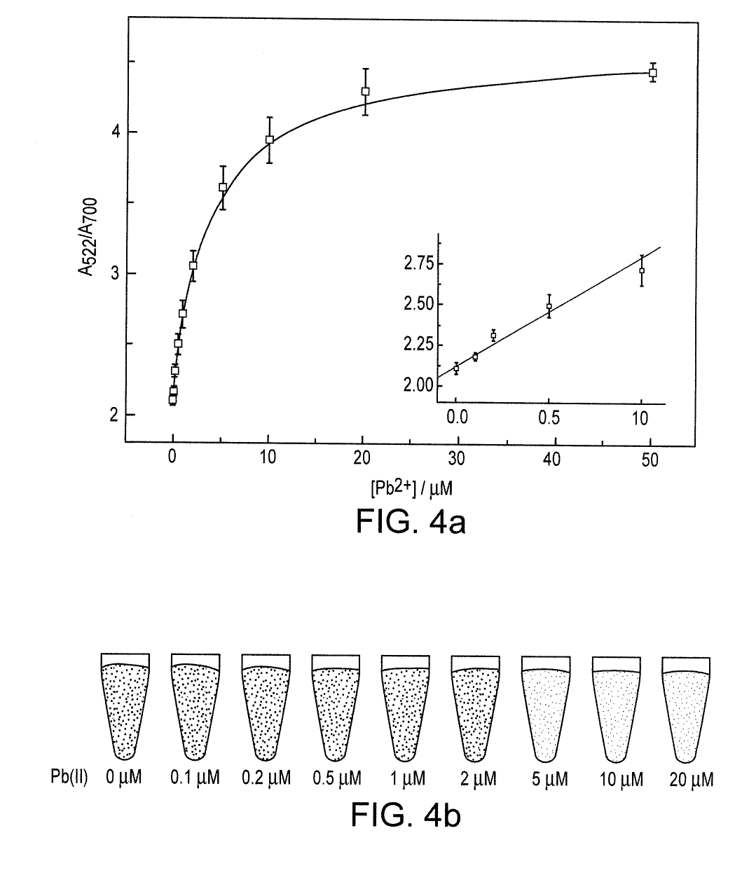

To test this hypothesis, the same label-free colorimetric sensing as shown above was carried out except that the reaction was at a pH of 5.5 using 10 mM 2-(N-morpholino)ethanesulfonic acid (MES) buffer. As shown in FIG. 4, the dynamic range was shifted to 120 nM-20 μM. Since both citrate and DNA contain functional groups that can be protonated, pH may affect the AuNP aggregation dynamics, as reported previously. A calibration curve should therefore be obtained at each specific pH in order to achieve accurate quantifications at different pHs. Furthermore, since the Pb²⁺ dependent cleavage reaction can be quenched by addition of EDTA, it is also possible to further tune the dynamic range simply by changing the reaction time. This dynamic range tunability allows accurate quantification of analytes over different concentration ranges without the need to develop new sensors.

### Experimental

Gold nanoparticles (13 nm in diameter) were prepared by sodium citrate reduction of HAuCl₄ following a procedure reported previously. All HPLC-purified DNA samples were purchased from Integrated DNA Technologies Inc. (Coralville, Iowa). A UV-V is spectrophotometer (Hewlett-Packard 8453) was used to check the exact concentration of DNAzyme strand 17E(8) and substrate strand (8)17S. Based on the measured concentration, (8)17S (4 μL, 100 μM) strand and an equal amount of 17E(8) strand were mixed in 100 μL buffer solution containing 100 mM NaCl and 10 mM Iris-HCl, pH 7.2 in a 0.6 mL micro centrifuge tube. After vortexing, the sample was heated up to 80° C. and cooled down to room temperature in one hour and thirty minutes. The hybridization solution volume can be increased according to the needs of the experiment.

After the cooling, 107 μL of solution containing the hybridized substrate and enzyme strand were transferred into a new tube. Pb²⁺ was added to the tube and left to react for 6 minutes. In order to quench the Pb²⁺ dependent cleavage reaction, 16 μL of a mixture solution were added to the same tube. The mixture solution contained 3.72 μL of a 200 mM EDTA solution, 5 μL of a 2 M NaCl solution, 0.2 Tris solution (0.2 μL 0.5 M) and Millipore water (7.08 μL).

AuNPs (76 μL of a 10 nM solution) were transferred to the tube containing DNA, and the solution showed a color change corresponding to the concentration of lead in the solution. The color change could be monitored by naked eye or by plasmon peak shift in UV-vis spectra. To show the tunability of the detection range of the sensor, the same amount of DNAzyme complex was formed in a MES (2-(N-morpholino)ethanesulfonic acid) buffer solution at a pH of 5.5. The buffer solution had a NaCl concentration of 100 mM and a MES concentration of 10 mM. The reaction was quenched with 16 μL of a mixture solution containing 3.72 μL of a 200 mM EDTA solution, 5 μL of a 2M NaCl solution, 1.2 μL of a Tris base solution, and 6.08 μL of Millipore water.

### Example 2

### Comparison of Uranyl (UO₂²⁺) Detection Using a Nucleic Acid Enzyme Directed Disassembly Sensor and a Label-Free Sensor

Two sensors for the colorimetric detection of uranyl were compared in various aspects. The first sensor was a traditional sensor based on nucleic acid enzyme directed disassembly of aggregates. The second was a label-free sensor based on the salt induced aggregation of label-free particles.

Nucleic Acid Enzyme Directed Disassembly Uranyl Sensor

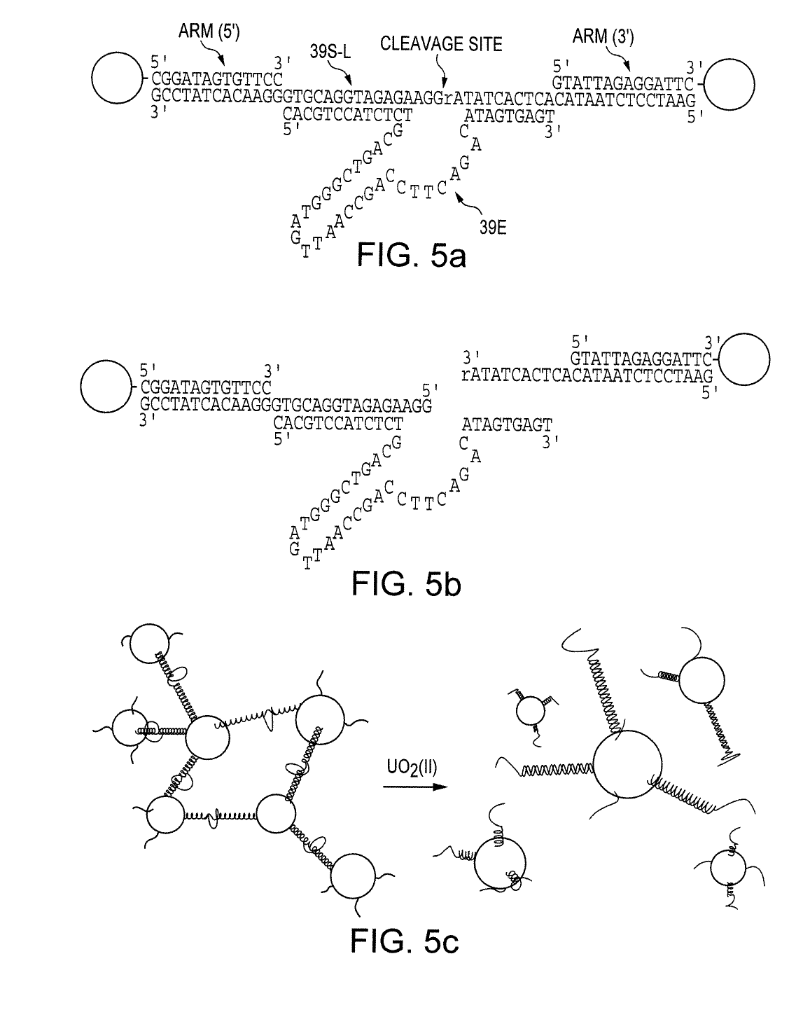

A uranyl specific DNAzyme was used to assemble oligonucleotide functionalized AuNPs to form purple colored aggregates as shown in (FIG. 5). The substrate strand is elongated on both ends (39S-L) to hybridize with oligonucleotides functionalized on AuNPs. After annealing the substrate strand (39S-L), enzyme strand (39E), and AuNPs functionalized with Arm (5′) and Arm (3′) DNA strands, AuNP aggregates are formed. Heating the system above the melting temperature results in disassembly of the gold nanoparticle aggregates due to dehybridization of the arm strands (Arm (5′) and Arm (3′)) from substrate strand (39S-L) which are 13 and 14 mer long, respectively (FIG. 5A). In the presence of uranyl, however, the substrate strand will be cleaved, which makes the 9 base pairs between the cleaved RNA site and 3′ end of the enzyme strand (39E) the weakest linkage in the system (FIG. 5B). This creates the difference in the melting temperatures for samples with or without uranyl, allowing for uranyl sensing.

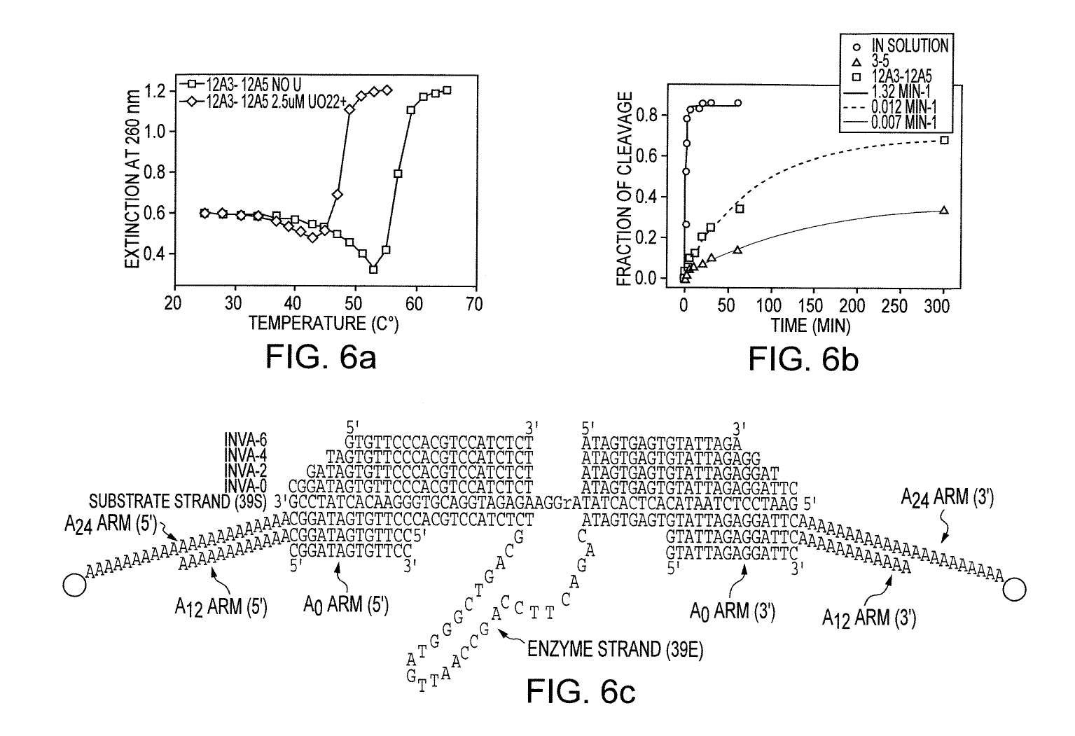

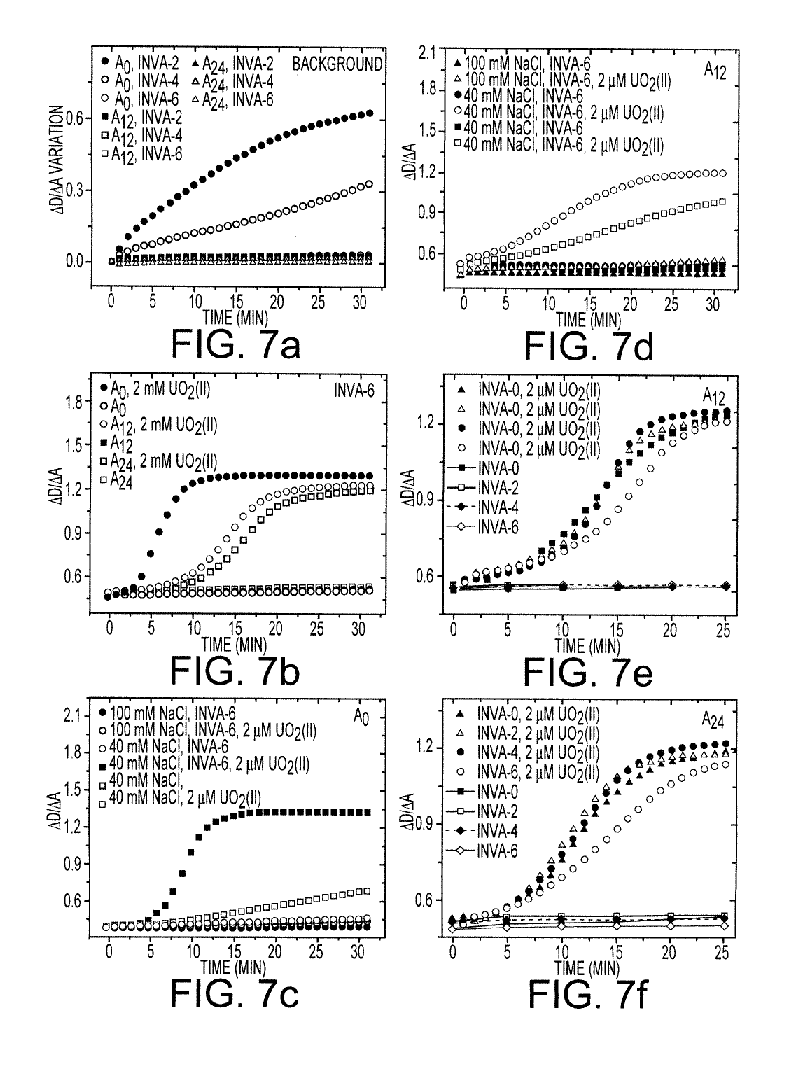

In order to measure the melting temperature of AuNP aggregates, AuNP aggregates were prepared and the melting temperatures of the AuNPs aggregates (A₁₂ aggregates, explained below) were measured in the absence and presence of uranyl (FIG. 6A). 2.5 μM of uranyl was added to one sample, and the sample was allowed to sit at room temperature overnight. A UV-vis spectrometer was used to monitor the extinction change of the samples at 260 nm. An increase in the extinction indicates melting of the aggregates.

As shown in FIG. 6A, in 300 mM NaCl solution, the sample with added uranyl had a melting temperature of ˜47° C. while the sample without added uranyl had a higher melting temperature of ˜57° C. Since there is a melting temperature difference of 10° C. between the two samples, heating the samples to a temperature between the two melting temperature, for example 50° C., will induce disassembly of the aggregates of the sample with added uranyl but not the sample without added uranyl.

Lower NaCl concentrations were investigated in order to lower the melting curve, but 40 mM NaCl was the lowest concentration to obtain stable AuNP aggregates, resulting in a melting temperature of ˜30° C. for the uranyl treated sample and ˜40° C. for the untreated sample. Since heating the sample is not very practical, two invasive DNA strands which are complementary to the both the 5′ and the 3′ ends of substrate strand can facilitate release of cleaved strands, and were used to induce disassembly at room temperature (FIG. 6C).

To study the kinetics of DNA cleavage, a ³²P assay was carried out by labeling the 39S-L strands with ³²P and using polyacrylamide gel electrophoresis (PAGE) (FIG. 6B). The results show that the kinetics in the aggregates (blue curve) was ˜200 times slower than that in solution (black curve). Insertion of a 12-mer poly-A spacer on the 5′ end of the Arm (5′) strand and the 3′ end of the Arm (3′) strand increased the rate by ˜100%; nevertheless, 5 hours were required to obtain 60% cleavage. Therefore, arm strands with different lengths of poly-A spacers were investigated to obtain the fastest rate of the cleavage reaction in the aggregates.

In order to find the optimum design of the nucleic acid enzyme sensor, invasive DNA with different lengths (Inva-0, Inva-2, Inva-4, and Inva-6) and arm strands with different poly-A spacers (0-mer, 12-mer, and 24-mer) were investigated. The design of DNA-disassembly sensors including invasive DNA strands and poly-A spacers and the sequences of the strands used in the study are depicted in FIG. 6C.

Optimization of the Sensor

To demonstrate the effect of invasive DNAs on disassembly of AuNPs aggregates, the disassembly of AuNP aggregates with arm strands containing O-mer, 12-mer, and 24-mer poly-A spacers were investigated in the presence of invasive DNAs (FIG. 7A). A UV-vis spectrometer was used to record the plasmon peak shift of the AuNPs, with the integration ratio between 490 nm to 540 nm and 550 nm to 700 nm chosen to monitor the color change. A lower ratio corresponds to aggregation of the AuNPs (blue color) while higher ratio corresponds to dispersed AuNPs (reddish color). AuNPs aggregates with 36-mer poly-A spacers were not investigated because no aggregation occurred when they were used.

Invasive DNA dependent disassembly occurred when AuNP aggregates without a poly-A spacer (A₀ aggregates) was used with Inva-2 and Inva-4, while disassembly was negligible with Inva-6. On the other hand, there was no invasive DNA dependent disassembly when AuNP aggregates with 12-mer poly-A (A₁₂ aggregates) or 24-mer poly-A spacers (A₂₄ aggregates) were used, regardless of the length of the invasive DNAs.

In order to determine which AuNP aggregates work best with invasive DNA in the presence of uranyl, A₀, A₁₂, and A₂₄ aggregates were investigated with Inva-6 (FIG. 7B). Inva-6 was chosen over Inva-2 or Inva-4 because of the negligible increase in background for A₀ aggregates. Surprisingly, A₀ aggregates showed the fastest disassembly kinetics with Inva-6, while A₁₂ and A₂₄ aggregates were both significantly slower. In the case of A₀ aggregates, disassembly started just 3 minutes after uranyl was added and saturated in about 10-15 minutes, while both A₁₂ and A₂₄ aggregates took about 25-30 minutes to disassemble. What was observed here differs from the result of the ³²P assay, which showed that poly-A spacers in the arm strands can help uranyl induced cleavage.

Since the only difference in the system is the presence of invasive DNA, it was suspected that it is the invasive DNA that makes the large difference in the kinetics of the systems. In order to prove this hypothesis, the disassembly kinetics of AuNP aggregates with and without Inva-6, in the presence of 2 μM uranyl, were compared. The disassembly of A₀ aggregates was very slow in the absence of Inva-6, but was very much accelerated when the invasive DNA was used (FIG. 7C). In contrast, even though the disassembly of A₁₂ aggregates was approximately 2 times faster than A₀ aggregates in the absence of Inva-6, its disassembly kinetics did not increase significantly with invasive DNA (FIG. 7D). We therefore concluded that Inva-6 works the most effectively in A₀ aggregates.

Sensitivity and Selectivity of the Nucleic Acid Enzyme Directed Disassembly Uranyl Sensor

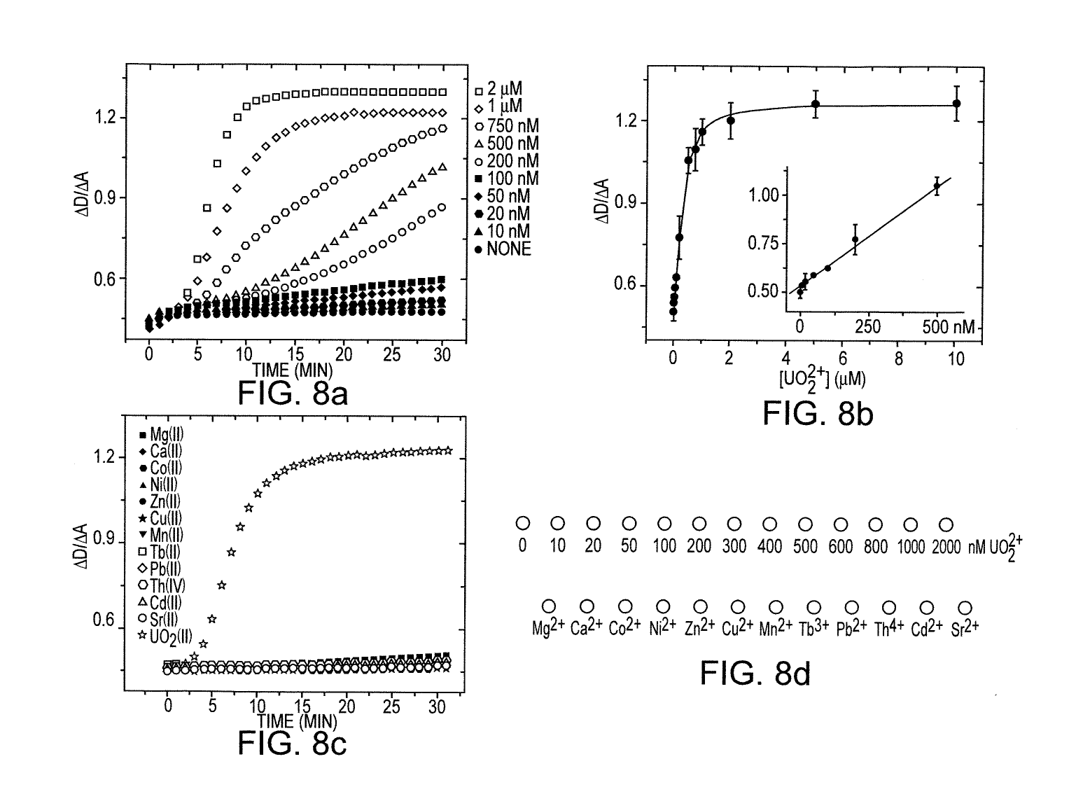

Through the optimization process, it was discovered that A₀ aggregates had the fastest disassembly kinetics when Inva-6 was used as the invasive DNA. In order to check the sensitivity of the uranyl dependent nucleic acid enzyme directed disassembly uranyl sensor, the plasmon resonance peak shift of AuNPs was monitored by UV-vis for 30 minutes; the kinetics of the reaction are shown in (FIG. 8A), based on the integration ratio between 490 nm to 540 nm and 550 nm to 700 nm at various uranyl concentrations. The integration between 490 nm to 540 nm, and 550 nm to 700 nm, represents dispersed (ΔD) and aggregated (ΔA) states of AuNPs, respectively. Nucleic acid enzyme directed disassembly based sensors can have extinction ratios (Abs₅₂₂ₙₘ/Abs₇₀₀ₙₘ) in the range of 1.5˜2 to values greater than 15. But since the absorption at 700 nm can be as low as 0.01˜0.03 after full reaction, a small difference in absorption at 700 nm can make a big difference in the extinction ratios, resulting in significantly increased error.

Therefore the integration ratio method is better than the extinction ratio (Abs₅₂₂ₙₘ/Abs₇₀₀ₙₘ) method to quantify the kinetics because it includes the absorption in the range of 550 nm to 700 nm to represent the aggregated state. The calibration curve based on the integration ratio of samples measured after 30 minutes of reaction is shown in FIG. 8B. Based on the calibration curve, the detection limit of the DNA directed disassembly based uranyl sensor is as low as 50 nM, and the calibration curve saturated at 2 μM. The image of color change is shown in FIG. 8D.

To investigate the selectivity of the DNA directed disassembly sensor, plasmon resonance peak shifts of the AuNPs were monitored by UV-vis for 30 minutes and the integration ratio change was compared for various metal ions, including uranyl (FIG. 8C). Only the sample with uranyl showed a change in plasmon shift, which means that the sensor has selectivity in uranyl; the color of the sensor solutions with several metal ions including uranyl are shown in FIG. 8D.

Label-Free Sensor

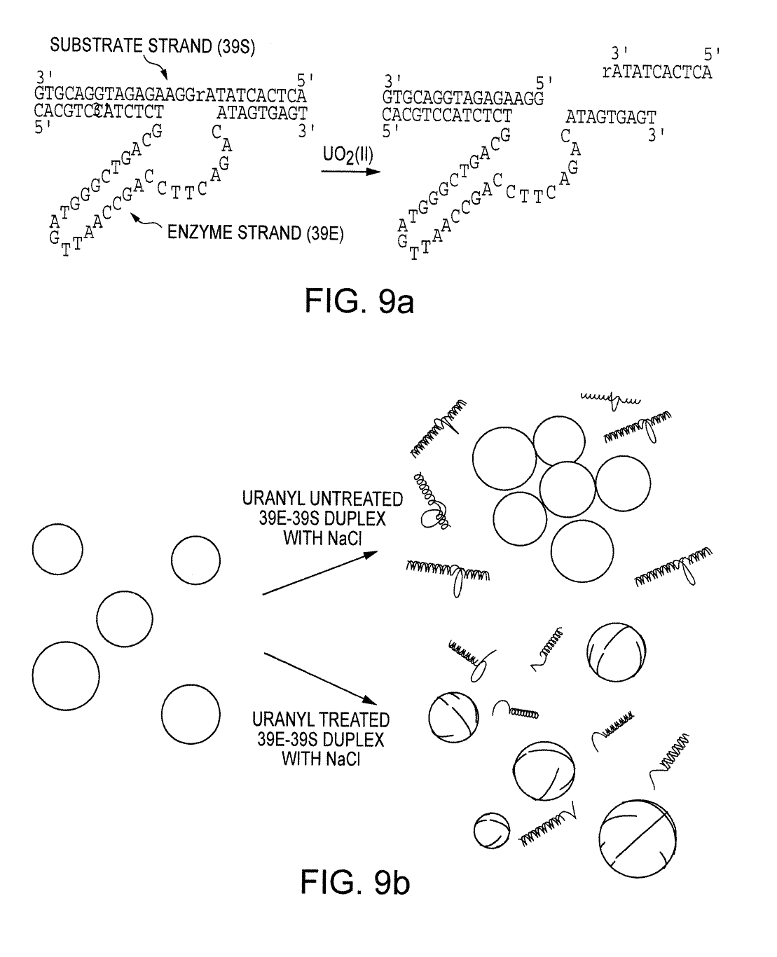

The scheme of the investigation is illustrated in FIG. 9. A uranyl cleavable substrate-nucleic acid enzyme complex was first prepared separately and reacted with uranyl. In the presence of uranyl, the substrate strand (39S) should be cleaved and 10-mer ssDNA released, which can then be absorbed on the AuNPs to prevent the salt-induced aggregation. In the absence of uranyl, however, the complex should remain double stranded and will not interact with the AuNPs, resulting in AuNP aggregation due to the screening effect from salts and causing a color change from red to blue.

Stability of AuNPs with the Addition of NaCl and ssDNA

In most of the reported salt-induced aggregation colorimetric sensors, ssDNA is absorbed on AuNPs surfaces first and salt is subsequently added to induce the color change. 24-mer dsDNA has been reported to remain hybridized for about 10 minutes in an Au colloid without NaCl, while introduction of a single mismatch will decrease the stability of the dsDNA and cause dehybridization in 5 min. Nucleic acid enzyme based systems, however, contain a very sizable mismatch between the enzyme strand and substrate strand, which causes dehybridization of the complex within seconds in the absence of salts.

Therefore, unlike previously reported studies, a DNA solution must be added to the AuNP solution together with a sufficient ionic strength to keep the complex hybridized. In this case, since the stability of the AuNPs is determined by the competition between the ssDNA absorption on the AuNPs and electrostatic screening caused by salt or salts introduced to the AuNP solution at the same time, it was important to investigate whether DNA can still be absorbed on AuNPs effectively and prevent aggregation in the presence of salts.

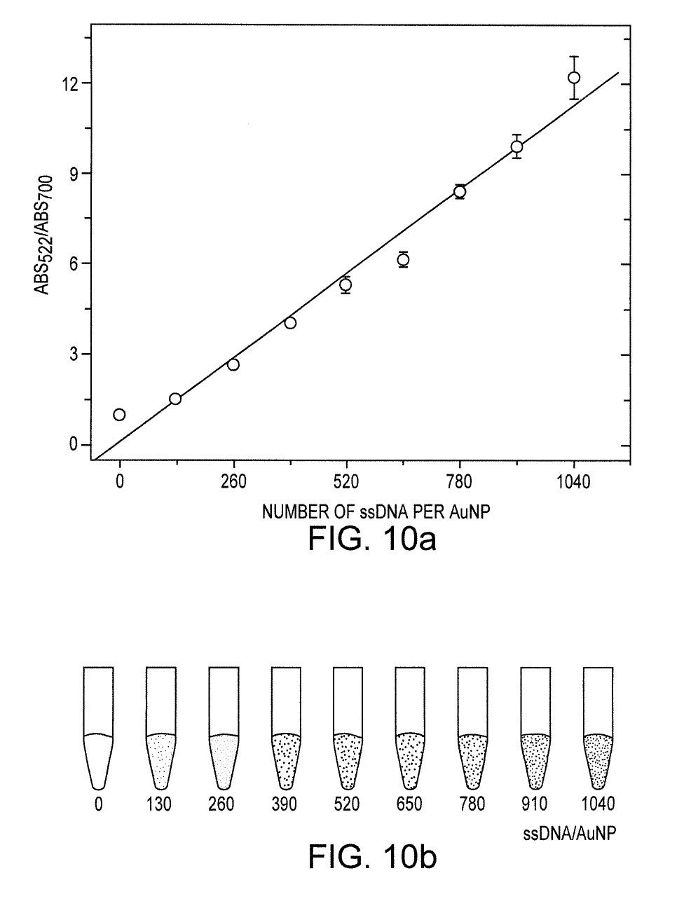

In order to investigate whether AuNPs can still be stabilized by ssDNA in the presence of NaCl, 10-mer ssDNA was chosen as a model DNA strand to simulate the protective effect of the cleaved ssDNA from the substrate. Different amounts of 10-mer ssDNA in 300 mM NaCl/10 mM MES (pH 5.5) were added to AuNP solutions and the color change was monitored (FIG. 10A) based on the extinction ratio between 522 nm and 700 nm. As the extinction ratio of the NaCl-induced aggregation sensor is only in the range of 1 to 5, there is no substantial error introduced by the low absorption at 700 nm. As absorption at 522 nm and 700 nm are equally weighted, this method is preferred over the integration ratio method.

The concentration of NaCl in the final solution was 0.1 M. The extinction ratio (Abs₅₂₂ₙₘ/Abs₇₀₀ₙₘ) of AuNPs is linearly dependent on the amount of DNA at 0.1 M NaCl. This shows that ssDNA can still stabilize AuNPs even though it has been introduced to the AuNP solution at the same time as the NaCl. The extinction ratio reached 11 when about 1000 equivalents of ssDNA were used per one AuNP. Since the extinction ratio change of from 1 to 5 is sufficient for detection, 500 equivalents of ssDNA per one AuNP were used in the following experiments since this quantity was sufficient to stabilize the AuNPs and produce a color change from blue to red.

Quenching Uranyl Dependent Cleavage Reaction by Shifting pH

Hybridization of the substrate and enzyme strand was carried out at a pH 5.5, where the uranyl dependent cleavage reaction is most active. Since the uranyl dependent cleavage reaction takes place very quickly, if the reaction cannot be effectively stopped during the measurements, a large error will result, making the sensor impractical. Since the biochemical investigation of the uranyl specific DNAzyme showed that its activity is highly pH dependent, with the activity peak occurring around pH 5.5 and a dramatic decrease of activity at either higher or lower pH, it was hypothesized that the DNAzyme might not be active at a pH of 8. Therefore, to quench the reaction, small amounts of concentrated Tris(2-Amino-2-(hydroxymethyl)propane-1,3-diol) base solution was added to the solution containing the complex to shift the pH from 5.5 to about 8.

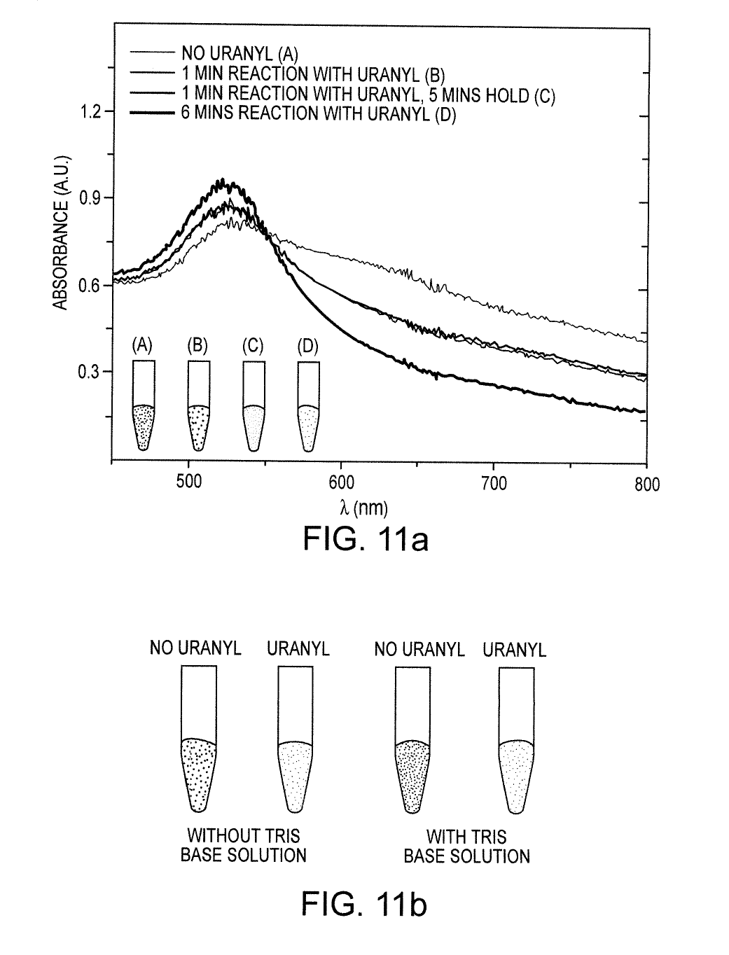

UV-vis spectra results showing the quenching effect of the uranyl-induced cleavage reaction using Tris base solution, are shown in (FIG. 11). When the complex was added to the AuNP solution without addition of uranyl, AuNPs aggregated, showing an extinction ratio of ˜1.4. On the other hand, when the complex was treated with 500 nM uranyl for 6 minutes, AuNPs remained dispersed, showing an extinction ratio of ˜4. When the reaction was quenched by adding Tris base solution 1 minute after the uranyl-induced reaction followed by addition to the AuNP solution, AuNPs were less dispersed, showing an extinction ratio of ˜2.2. This means that the uranyl-induced cleavage reaction had been quenched.

To ensure that the quenching reaction was complete and no further uranyl-induced reaction took place afterwards, a control experiment was carried out at 5 minutes intervals between the uranyl-induced cleavage reaction (1 minute) and the mixing in of the AuNP solution. Even though there was a 5 minute interval between the quenching and the mixing in of the AuNP solution, no further uranyl-induced cleavage reaction took place. This indicates that the Tris base solution could quench the reaction very effectively and in time. Furthermore, the Tris base solution aided in aggregating the AuNPs more effectively, which also resulted in lower background.

Sensitivity and Selectivity of NaCl-Induced Aggregation Based Sensor to Uranyl

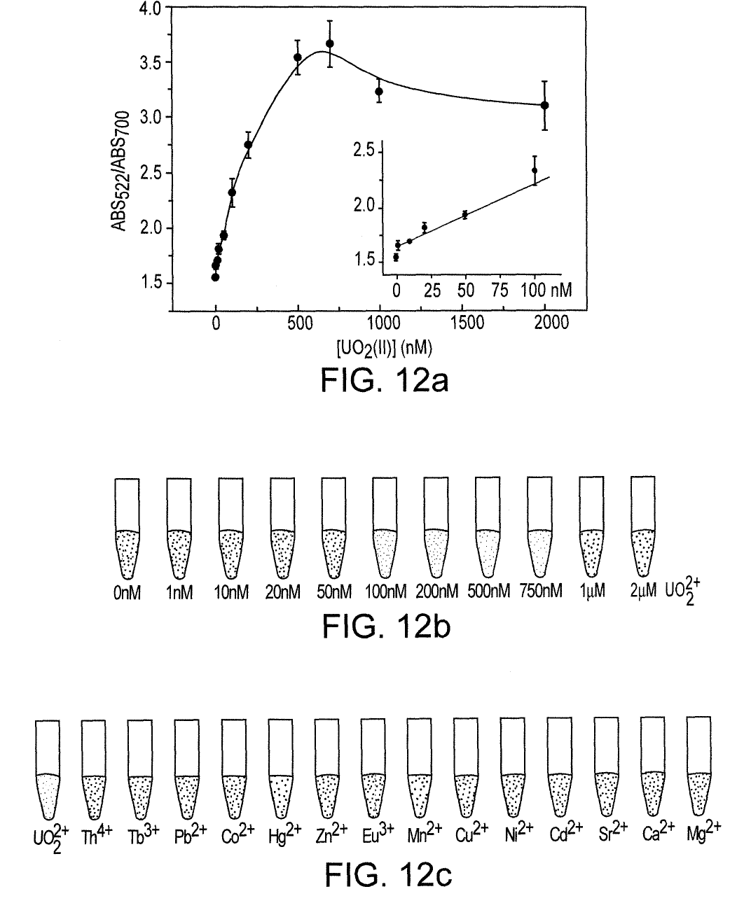

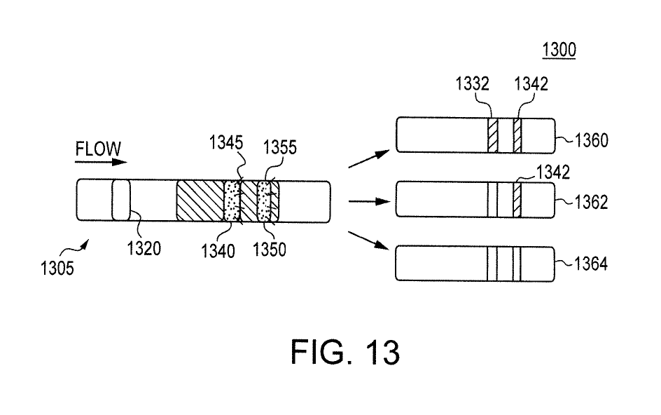

In order to measure the sensitivity of the uranyl dependent NaCl-induced aggregation sensor, plasmon resonance peak shifts of AuNPs were monitored by UV-vis, and the extinction ratio between 522 nm and 700 nm was compared at various uranyl concentrations (FIG. 12B). The detection limit was as low as 1 nM and the linear fit range from 1 nM to 500 nM. The calibration curve saturated at 400 nM, similar to the fluorescence-based uranyl sensor. Since the uranyl dependent cleavage reaction was carried out in a concentrated DNAzyme solution under optimized conditions, followed by the addition of AuNPs, the uranyl dependent cleavage reaction can occur very efficiently, which helps to maintain high sensitivity. Furthermore, as reacted DNA solution containing NaCl was added to AuNP solution after quenching, the color of AuNP solution change took place immediately and did not change much afterwards. The color change is shown in FIG. 12A. Since uranyl dependent cleavage reaction can easily be quenched by shifting pH from 5.5 to 8, it is possible to tune the dynamic range simply by changing the reaction time.

Furthermore, as reacted DNA solution containing NaCl was added to AuNP solution after quenching, the color of AuNP solution change took place immediately and did not change much afterwards. The color change is shown in FIG. 12A. Since the uranyl dependent cleavage reaction can easily be quenched by shifting pH from 5.5 to 8, it is possible to tune the dynamic range simply by changing the reaction time.

To investigate the selectivity of NaCl-aggregation sensor, several metal ions including uranyl were added to the sensor solution. The resulting color changes are shown in FIG. 12C. The results clearly show that the sensor responds only in the presence of uranyl, proving that the sensor has good selectivity.

Comparison Between the Nucleic Acid Enzyme Directed Disassembly Sensor and the Label-Free Sensor Based on NaCl-Induced Aggregation

Both colorimetric methods were demonstrated to successfully detect uranyl, providing the motivation to compare the two sensors in various aspects, as reported in Table 1:

TABLE 1
Comparison between DNA-disassembly and
NaCl-aggregation colorimetric sensors

| | Sensors | | | |
|---|---|---|---|---|
| | Assembly-disassembly | | NaCl aggregation | |
| | based | | based | |
| Detection range | 50 nM-2 μM | | 1 nM-2 μM | |
| Detection limit | 50 | nM | 1 | nM |
| Linear range | 50-500 | nM | 1-500 | nM |
| Saturation point | 2 | μM | 1 | μM |
| Working time | 30 | minutes | 6 | minutes |
| Working | Room temperature | | Room temperature | |
| temperature | | | | |
| Operation step | 1 | step | 3 | steps |

| | | |
|---|---|---|
| Quenching | No | Possible (by shifting pH) |
| Error bar | ~10% of the signal | ~10% of the signal |
| | change when saturated | change when saturated |
| Color Change | Purple to red (in the | Red to blue (in the |
| | presence of analyte) | absence of analyte) |
| Type | Turn on | Turn off |
| Stability of | Stable | Less stable |
| AuNPs | | |

In terms of performance, as the nucleic acid enzyme directed disassembly sensor colorimetric sensing method depends on uranyl induced cleavage of substrate strands in the aggregated state, a certain amount of both uranyl and time are necessary, which is the reason for the relatively high detection limit (50 nM) and slow kinetics (30 minutes). In contrast, the uranyl-induced cleavage reaction of the NaCl-aggregation sensor is carried out separately, and the AuNPs solution is added afterwards. The NaCl-aggregation sensor is therefore much more sensitive (1 nM) and the reaction much faster (6 minutes).

The quenching step of the NaCl-aggregation sensor is also advantageous, since the reaction can be stopped and the color change remains stable after adding AuNPs, which renders the sensor easier to control and detect. Furthermore, the NaCl-aggregation sensor has error bars comparable to DNA-disassembly sensor (˜10% of the total signal change).

Materials and Methods

Oligonucleotides and Reagents

All Oligonucleotides were purchased from Integrated DNA Technologies Inc. (Coralville, Iowa). DNAzyme strand (39E) and both substrate strands (39S-L for DNA-disassembly sensor and 39S for NaCl-aggregation sensor) were purified by HPLC by the manufacturer. The arm strands with thiolate modifications and invasive DNA strands were desalted according to standard methods. HAuCl₄ (99.999%) and sodium citrate dehydrate (>99%) were purchased from Aldrich and used without further purification.

Preparation and Functionalization of Gold Nanoparticles

Gold nanoparticles (13 nm diameter) were synthesized by reduction of HAuCl₄ with sodium citrate, and the AuNP-DNA conjugates were prepared following the published protocol (22). In order to activate thiol modification on the Arm (5′) strand, 9 μL of 1 mM Arm (5′) strand, 1 μL of 500 mM pH 5.5 MES buffer, and 1.5 μL of 10 mM TCEP solution were mixed in a microcentrifuge tube and allowed to stand for one hour. In a separate tube, a parallel experiment was carried out to activate thiol modification on the Arm (3′) strand.

At the same time, two scintillation vials were incubated in fresh 10 M NaOH solution for an hour and rinsed repeatedly with distilled water and then with Millipore water in order to prevent AuNP from sticking to the glass surface of the vials.

In order to attach the Arm (5′) DNA strand to the AuNPs, 3 mL of 13 nm AuNP solution were placed in one scintillation vial and activated. 9 μL Arm (5′) strand were added. Gentle shaking followed, and the resulting mixture was allowed to react overnight in a dark place. In the other scintillation vial, another 3 mL of 13 nm AuNP solution were mixed with activated 9 μL Arm (3′) strand and subjected to the same treatment outlined above for the Arm (5′) DNA strand.

300 μL of 1 M NaCl and 15 μL of 500 mM Tris-acetate buffer (pH 7.6) were added to each vial on the next day, and the vials were subjected to gentle shaking and allowed to stand in a dark place for another night.

In order to prepare the DNA-disassembly sensor, DNA functionalized AuNPs were first purified to remove free DNA in the AuNP solution. 500 μL of AuNPs functionalized with the Arm (5′) strand and an equivalent amount of AuNP functionalized with the Arm (3′) strand were placed in two 1.5 mL micro-centrifuge tubes, respectively, and centrifuged at 13.2 krpm for 15 minutes. Supernatants in both solutions were then replaced with 500 μL of fresh 50 mM MES (pH 5.5) 100 mM NaCl solution. The purification process was repeated, and the supernatant was replaced with 250 μL of 50 mM MES (pH 5.5) 300 mM NaCl buffer. After mixing the two AuNP solutions, 10 μL of 10 μM elongated substrate strand (39S-L), and 20 μL of 10 μM enzyme strand (39E) were added and annealed from 55° C. to room temperature for about 1 hour.

The color of the AuNP solution changed from red to blue, which shows that DNA-directed assembly of AuNPs had taken place. The AuNP aggregates were centrifuged with a micro-centrifuge for about a minute and the supernatant was replaced with 120 μL of 300 mM NaCl 50 mM MES (pH 5.5) solution to remove free DNA (39S-L and 39E) from the sensor solution.

Activity Assays

To prepare aggregates containing ³²P-labeled substrate, ˜0.1% of ³²P-labeled substrates (with respect to the total substrate amount) were added, while keeping other conditions the same. ³²P-labeled aggregates were added to a solution containing 1 μM uranyl, 300 mM NaCl and 50 mM MES, pH 5.5. Aliquots were taken out at designated time points and quenched in a solution containing 8 M urea and 200 mM EDTA. The quenched aliquots were heated to 60° C. to fully release substrate strands from aggregates and then loaded o a 20% denaturing polyacrylamide gel. ³²P-labeling and procedures for single-turnover solution phase activity assays were the same as reported elsewhere.

Uranyl Detection

In order to detect uranyl using the DNA-disassembly sensor, 426 μL of 50 mM MES 35.5 mM NaCl buffer (pH 5.5), 4.5 μL of 1 mM Inva-6 (5′), 4.5 μL of 1 mM Inva-6 (3′) were mixed and 15 μL of sensor solution in 50 mM MES 300 mM NaCl (pH 5.5) were added just prior to UV-vis measurement. Uranyl was added 1 minute after the measurement had started and the reaction was allowed to carry on for 30 minutes. A calibration curve was made based on the data collected on the 31st minute after the 30 minutes of reaction-time.

NaCl-Aggregation Sensor

AuNPs Stabilized by ssDNA in the Presence of NaCl

Different amounts (from 0 μL to 8 μL) of 10mer DNA (5′-CAT GCT ACT G-3′, 100 μM) were added to 70 μL 300 mM NaCl 10 mM MES buffer solution (pH 5.5) in a 0.6 mL microcentrifuge tube. A mixture of 1.19 μL of 500 mM Tris base solution and an appropriate amount of Millipore water was added to obtain a total volume of 134 μL. After vortexing, 76 μL 10 nM Au nanoparticles (13 nm) were added and the surface plasma absorption spectrum was collected with a UV-vis spectrometer.

Sensor Preparation and Uranyl Detection

UV-Vis spectrometry was used to measure the exact concentration of 39E and 39S strands. This process is very important because a very small number of unhybridized ssDNA strands can still stabilize AuNP and increase the background. Based on the measured concentration, 4 μL of 100 μM solution of 39S strand and an equal amount of 39E strand were mixed in 70 μL 300 mM NaCl 10 mM MES buffer solution (pH 5.5) in a 0.6 mL microcentrifuge tube. After vortexing, the sample was heated to 80° C. and allowed to cool down to room temperature for 90 minutes. The hybridization solution can be increased by scaling up its volume.

Subsequently, 77 μL of solution containing hybridized DNAzyme and enzyme strand were transferred into a new tube and cleaved by uranyl for 6 minutes. In order to quench the uranyl-dependent cleavage reaction, a mixture of 1.19 μL of 500 mM Tris base solution and 56 μL Millipore water was added to the same tube and the tube was vortexed quickly, resulting in the pH shifting from 5.5 to 8.76 μL of 10 nM Au nanoparticles (13 nm) were transferred to the tube containing DNA. The solution showed a color change corresponding to the concentration of uranyl in the solution. The color change could be monitored by eye or by plasmon peak shift in the UV-vis spectra.

### REFERENCES

A. K. Brown, J. Li, C. M. B. Pavot, Y. Lu, Biochemistry 2003, 42, 7152.
A. P. Alivisatos, K. P. Johnsson, X. Peng, T. E. Wilson, C. J. Loweth, M. P. Bruchez, Jr, P. G. Schultz, Nature 1996, 382, 609.
A. P. F. Turner, Science 2000, 290, 1315.
Abbasi, S. A., Int. J. Environ. Anal. Chem. 1989, 36, 163-172.
Allara D, Nuzzo R. (1985) Spontaneously organized molecular assemblies. 1. Formation, dynamics and physical properties of n-alkanoic acids adsorbed from solution on an oxidized aluminum surface. Langmuir 1: 45-52.
Andreola, M.-L., Pileur, F., Calmels, C., Ventura, M., Tarrago-Litvak, L., Toulme, J.-J. & Litvak, S. (2001). DNA aptamers selected against the HIV-1 RNase H display in vitro antiviral activity. Biochemistry 40: 10087-94.
Arnez, J. G.; Steitz, T. A., Biochemistry 1994, 33, 7560-7567.
Beebe T, Rabke-Clemmer C, (1995) Thiol labeling of DNA for attachment to gold surfaces. U.S. Pat. No. 5,472,881 USA.
Been, M. D. & Wickham, G. S. (1997). Self-cleaving ribozymes of hepatitis delta virus RNA. Eur. J. Biochem. 247: 741-53.
Biroccio A, Hamm J, Incitti I, De Francesco R, Tomei L. (2002) Selection of RNA aptamers that are specific and high-affinity ligands of the hepatitis C virus RNA-dependent RNA polymerase. J Virol 76: 3688-3696.
Blake, R. C., II; Pavlov, A. R.; Khosraviani, M.; Ensley, H. E.; Kiefer, G. E.; Yu, H.; Li, X.; Blake, D. A., Bioconjug. Chem. 2004, 15, 1125-1136.
Bloomfield, V. A.; Crothers, D. M.; Tinoco, I., Jr, Section Title: General Biochemistry 2000, 800.
Bock, L. C., Griffin, L. C., Latham, J. A., Vermaas, E. H. & Toole, J. J. (1992). Selection of single-stranded DNA molecules that bind and inhibit human thrombin. Nature (London) 355: 564-6.
Boomer, D. W.; Powell, M. J., Anal. Chem. 1987, 59, 2810-2813.
Breaker R R, Joyce G F, Breaker R R, Joyce G F. (1995) A DNA enzyme with Mg(2+)-dependent RNA phosphoesterase activity; A DNA enzyme that cleaves RNA. Chem Biol; Chem Biol 2; 1: 223-229.
Breaker R R. (2002) Engineered allosteric ribozymes as biosensor components. Curr Opin Biotechnol 13: 31-39.
Breaker, R. R. (1997). DNA enzymes. Nat. Biotechnol. 15: 427-431.
Breaker, R. R. (1999). Catalytic DNA: in training and seeking employment. Nat. Biotechnol. 17: 422-423.
Breaker, R. R. (2000). Making catalytic DNAs. Science (Washington, D.C.) 290: 2095-6.
Brina, R.; Miller, A. G., Anal. Chem. 1992, 64, 1413-1418.
Brody E N, Gold L. (2000) Aptamers as therapeutic and diagnostic agents. J Biotechnol 74: 5-13.
Brown A, Pavot C, Li J, Lu Y. A lead-dependent DNAzyme with a two-step mechanism. submitted.
Brown, A. K.; Li, J.; Pavot, C. M. B.; Lu, Y., Biochemistry 2003, 42, 7152-7161.
Bruno J G, Kiel J L. (1999) In vitro selection of DNA aptamers to anthrax spores with electrochemiluminescence detection. Biosens Bioelectron 14: 457-464.
Bruno J G, Kiel J L. (2002) Use of magnetic beads in selection and detection of biotoxin aptamers by electrochemiluminescence and enzymatic methods. Biotechniques 32: 178-80, 182-3.
Buell P, Girard, 2003, Chemistry Fundamentals: An Environmental Perspective. Sudbury, M A: Jones & Bartlett.
Burdette, S. C.; Walkup, G. K.; Spingler, B.; Tsien, R. Y.; Lippard, S. J., J. Am. Chem. Soc. 2001, 123, 7831-7841.
Burgstaller, P. & Famulok, M. (1994). Isolation of RNA aptamers for biological cofactors by in vitro selection. Angew. Chem. 106: 1163-6 (See also Angew. Chem., Int. Ed. Engl., 994, 33(10), 084-7).
Burgstaller, P., Kochoyan, M. & Famulok, M. (1995). Structural probing and damage selection of citrulline- and arginine-specific RNA aptamers identify base positions required for binding. Nucleic Acids Res. 23: 4769-76.
Burke, D. H. & Gold, L. (1997). RNA aptamers to the adenosine moiety of S-adenosyl methionine: structural inferences from variations on a theme and the reproducibility of SELEX. Nucleic Acids Res. 25: 2020-4.
Burke, D. H., Hoffman, D. C., Brown, A., Hansen, M., Pardi, A. & Gold, L. (1997). RNA aptamers to the peptidyl transferase inhibitor chloramphenicol. Chem. Biol. 4: 833-43.
Burmeister, J., Von Kiedrowski, G. & Ellington, A. D. (1997). Cofactor-assisted self-cleavage in DNA libraries with a 3′-′5′-phosphoramidate bond. Angew. Chem., Int. Ed. Engl. 36: 1321-4.
C. A. Mirkin, R. L. Letsinger, R. C. Mucic, J. J. Storhoff, Nature 1996, 382, 607.
C. Tuerk, L. Gold, Science 1990, 249, 505; b) A. D. Ellington, J. W. Szostak, Nature 1990, 346, 818.
Cadwell R C, Joyce G F. (1992) Randomization of genes by PCR mutagenesis. PCR Methods Appl 2: 28-33.
Cadwell R C, Joyce G F. (1994) Mutagenic PCR. PCR Methods Appl 3: S136-40.
Cao Y, Jin R, Mirkin C A. (2001) DNA-modified core-shell Ag/Au particles. J Am Chem Soc 123: 7961-7962.
Carmi N, Shultz L A, Breaker R R. (1996) In vitro selection of self-cleaving DNAs. Chem. Biol 3: 1039-1046.
Carmi, N.; Balkhi, H. R.; Breaker, R. R., Proc. Natl. Acad. Sci. U.S.A. 1998, 95, 2233-2237.
Cech, T. R. & Herschlag, D. (1996). Group I ribozymes: substrate recognition, catalytic strategies, and comparative mechanistic analysis. Nucleic Acids Mol. Biol. 10:1-17.
Cech, T. R. (1993). Structure and mechanism of the large catalytic RNAs: group I and group II introns and ribonuclease P. In The RNA World (Gesteland, R. F. & Atkins, J. F., ed.), pp. 239-70, Cold Spring Harbor Laboratory Press, Cold Spring Harbor, N.Y.
Chaloin L, Lehmann M J, Sczakiel G, Restle T. (2002) Endogenous expression of a high-affinity pseudoknot RNA aptamer suppresses replication of HIV-1. Nucleic Acids Res 30: 4001-4008.
Chapman K B, Szostak J W. (1994) In vitro selection of catalytic RNAs. Curr Opin Struct Biol 4: 618-622.
Chen, P.; He, C., J. Am. Chem. Soc. 2004, 126, 728-729.
Chung N H, Tabata M, 2002, Talanta 58: 927-933.
Ciesiolka J, Gorski J, Yarus M. (1995) Selection of an RNA domain that binds Zn2+. RNA 1: 538-550.
Conaty J, Hendry P, Lockett T. (1999) Selected classes of minimised hammerhead ribozyme have very high cleavage rates at low Mg2+ concentration. Nucleic Acids Res 27: 2400-2407.
Conn M, Prudent J, Schultz P. (1996) Porphyrin Metallation Catalyzed by a Small RNA Molecule. J Am Chem Soc 118: 7012-7013.
Connell, G. J. & Yarus, M. (1994). RNAs with dual specificity and dual RNAs with similar specificity. Science (Washington, D.C.) 264: 1137-41.
Connell, G. J., Illangesekare, M. & Yarus, M. (1993). Three small ribooligonucleotides with specific arginine sites. Biochemistry 32: 5497-502.
Craft, E.; Abu-Qare, A.; Flaherty, M.; Garofolo, M.; Rincavage, H.; Abou-Donia, M., J. Toxicol. Environ. Health B Crit. Rev. 2004, 7, 297-317.
Cuenoud B, Szostak J W. (1995) A DNA metalloenzyme with DNA ligase activity. Nature 375: 611-614.
D. Faulhammer, M. Famulok, Angew. Chem. Int. Ed. 1996, 35, 2837.
D. S. Wilson, J. W. Szostak, Annu. Rev. Biochem. 1999, 68, 611.
Dai X, De Mesmaeker A, Joyce G F. (1995) Cleavage of an amide bond by a ribozyme. Science 267: 237-240.
Demers, L. M.; Oestblom, M.; Zhang, H.; Jang, N.-H.; Liedberg, B.; Mirkin, C. A., J. Am. Chem. Soc. 2002, 124, 11248-11249.
Derose, V. J. (2002). Two Decades of RNA Catalysis. Chemistry & Biology 9: 961-9.
E. Katz, I. Willner, Angew. Chem. Int. Ed. 2004, 43, 6042.
Earnshaw, Gait. (1998) Modified oligoribonucleotides as site-specific probes of RNA structure and function. Biopolymers 48: 39-55.
Ekland E H, Bartel D P. (1996) RNA-catalysed RNA polymerization using nucleoside triphosphates. Nature 382: 373-376.
Ekland E H, Szostak J W, Bartel D P. (1995) Structurally complex and highly active RNA ligases derived from random RNA sequences. Science 269: 364-370.
Elghanian, R.; Storhoff, J. J.; Mucic, R. C.; Letsinger, R. L.; Mirkin, C. A., Science 1997, 277, 1078-1080.
Ellington A D, Szostak J W. (1990) In vitro selection of RNA molecules that bind specific ligands. Nature 346: 818-822.
Ellington, A. D. & Conrad, R. (1995). Aptamers as potential nucleic acid pharmaceuticals. Biotechnol. Annu. Rev. 1: 185-214.
Ellington, A. D. & Szostak, J. W. (1992). Selection in vitro of single-stranded DNA molecules that fold into specific ligand-binding structures. Nature (London) 355: 850-2.
Famulok, M. & Huettenhofer, A. (1996). In Vitro Selection Analysis of Neomycin Binding RNAs with a Mutagenized Pool of Variants of the 16S rRNA Decoding Region. Biochemistry 35: 4265-70.
Famulok, M. & Szostak, J. W. (1992). Stereospecific recognition of tryptophan agarose by in vitro selected RNA. J. Am: Chem. Soc. 114: 3990-1.
Famulok, M. (1994). Molecular Recognition of Amino Acids by RNA-Aptamers: An L-Citrulline Binding RNA Motif and Its Evolution into an L-Arginine Binder. J. Am. Chem. Soc. 116: 1698-706.
Faulhammer D, Famulok M. (1997) Angew Chem Int Ed Engl 35: 2837-2841.
Faulhammer, D. & Famulok, M. (1996). The Ca2+ ion as a cofactor for a novel RNA-cleaving deoxyribozyme. Angew. Chem., Int. Ed. Engl. 35: 2837-41.
Frankforter G B, Cohen L, 1914, J. Am. Chem. Soc. 36: 1103-1134.
Frankforter G B, Fray F C, 1913, J. Phys. Chem. 17: 402-473.
Frankforter G B, Temple S, 1915, J. Am. Chem. Soc. 37: 2697-2716.
Fukusaki, E.-I., Kato, T., Maeda, H., Kawazoe, N., Ito, Y., Okazawa, A., Kajiyama, S.-I. & Kobayashi, A. (2000). DNA aptamers that bind to chitin. Bioorg. Med. Chem. Lett. 10: 423-5.
G. F. Joyce, Ann. Rev. Biochem. 2004, 73, 791.
G. K. Walkup, B. Imperiali, J. Am. Chem. Soc. 1996, 118, 3053.
Geiger, A., Burgstaller, P., Von Der Eltz, H., Roeder, A. & Famulok, M. (1996). RNA aptamers that bind L-arginine with sub-micromolar dissociation constants and high enantioselectivity. Nucleic Acids Res. 24: 1029-36.
Geyer C R, Sen D. (1997) Evidence for the metal-cofactor independence of an RNA phosphodiester-cleaving DNA enzyme. Chem Biol 4: 579-593.
Ginnings P M, Robbins D, 1930, Ternary systems: water, tertiary butanol and salts at 30° C., J. Am. Chem. Soc. 52: 2282-2286.
Giver, L., Bartel, D., Zapp, M., Pawul, A., Green, M. & Ellington, A. D. (1993). Selective optimization of the Rev-binding element of HIV-1. Nucleic Acids Res. 21: 5509-16.
Giver, L., Bartel, D. P., Zapp, M. L., Green, M. R. & Ellington, A. D. (1993). Selection and design of high-affinity RNA ligands for HIV-1 Rev. Gene 137: 19-24.
Gongalsky Konstantin, B., Environ. Monit. Assess. 2003, 89, 197-219.
Gould R F., 1972, Extractive and Azeotropic Distillation. Advances in Chemistry Series. No. 115. Washington: American Chemical Society.
Grabar K, Freeman R, Hommer M, Natan M. (1995) Preparation and characterization of Au colloid Monolayers. Anal. Chem. 67: 735-743.
H. L. Needleman, Human Lead Exposure, CRC Press, Boca Raton 1992.
H. Li, L. J. Rothberg, J. Am. Chem. Soc. 2004, 126, 10958.
H. Li, L. Rothberg, Anal. Chem. 2005, 77, 6229.
H. Li, L. Rothberg, Proc. Natl. Acad. Sci. U.S.A. 2004, 101, 14036.
H. Wei, B. Li, J. Li, E. Wang, S. Dong, Chem. Comm. 2007, 3735.
H. Yan, Science 2004, 306, 2048; e) Y. Lu, J. Liu, Acc. Chem. Res. 2007, 40, 315.
Haller, A. A. & Sarnow, P. (1997). In vitro selection of a 7-methyl-guanosine binding RNA that inhibits translation of capped mRNA molecules. Proc. Natl. Acad. Sci. U.S.A. 94: 8521-6.
Harada, K. & Frankel, A. D. (1995). Identification of two novel arginine binding DNAs. EMBO J. 14: 5798-811.
Hazarika, P.; Ceyhan, B.; Niemeyer, C. M., Angew. Chem., Int. Ed. 2004, 43, 6469-6471.
Hesselberth J, Robertson M P, Jhaveri S, Ellington A D. (2000) In vitro selection of nucleic acids for diagnostic applications. J Biotechnol 74: 15-25.
Hofmann H P, Limmer S, Hornung V, Sprinzl M. (1997) Ni2+-binding RNA motifs with an asymmetric purine-rich internal loop and a G-A base pair. RNA 3: 1289-1300.
Holeman, L. A., Robinson, S. L., Szostak, J. W. & Wilson, C. (1998). Isolation and characterization of fluorophore-binding RNA aptamers. Folding Des. 3: 423-31.
Homola, J.; Piliarik, M., Springer Series on Chemical Sensors and Biosensors 2006, 4, 45-67.
Horsley L H, 1911, Azeotropic Data-Ill. Advances in Chemistry Series. No. 116. American Chemical Society, Washington D.C.
http://www.epa.gov/safewater/contaminants/index.html
http://www.epa.gov/safewater/contaminants/index.html
Huang, C.-C.; Huang, Y.-F.; Cao, Z.; Tan, W.; Chang, H.-T., Anal. Chem. 2005, 77, 5735-5741.
Huizenga D E, Szostak J W. (1995) A DNA aptamer that binds adenosine and ATP. Biochemistry 34: 656-665.
Iler R. (1979) Chapter 6. In: anonymous (eds) The chemistry of silica. Wiley, New York.
Illangasekare M, Yarus M. (1997) Small-molecule-substrate interactions with a self-aminoacylating ribozyme. J Mol Biol 268: 631-639.
J. H. Lee, D. P. Wernette, M. V. Yigit, J. Liu, Z. Wang, Y. Lu, Angew. Chem. Int. Ed. 2007, 46, 9006.
J. J. Storhoff, A. A. Lazarides, R. C. Mucic, C. A. Mirkin, R. L. Letsinger, G. C. Schatz, J. Am. Chem. Soc. 2000, 122, 4640.
J. J. Storhoff, C. A. Mirkin, Chem. Rev. 1999, 99, 1849.
J. J. Storhoff, R. Elghanian, R. C. Mucic, C. A. Mirkin, R. L. Letsinger, J. Am. Chem. Soc. 1998, 120, 1959.
J. Li, W. Zheng, A. H. Kwon, Y. Lu, Nucleic Acids Res. 2000, 28, 481.
J. Li, Y. Lu, J. Am. Chem. Soc. 2000, 122, 10466.
J. Liu, A. K. Brown, X. Meng, D. M. Cropek, J. D. Istok, D. B. Watson, Y. Lu, Proc. Natl. Acad. Sci. U.S.A. 2007, 104, 2056.
J. Liu, Y. Lu, Adv. Mater. 2006, 18, 1667.
J. Liu, Y. Lu, Anal. Chem. 2003, 75, 6666.
J. Liu, Y. Lu, Angew. Chem. Int. Ed. 2006, 45, 90.
J. Liu, Y. Lu, Curr. Opion. Biotech. 2006, 17, 580.
J. Liu, Y. Lu, J. Am. Chem. Soc. 2003, 125, 6642.
J. Liu, Y. Lu, J. Am. Chem. Soc. 2007, 129, 8634.
J. Liu, Y. Lu, J. Am. Chem. Soc. 2007, 129, 9838; f) J. Liu, Y. Lu, Angew. Chem. Int. Ed. 2007, 46, 7587.
J. Liu, Y. Lu, J. Fluoresc. 2004, 14, 343.
J. Sharma, R. Chhabra, Y. Liu, Y. Ke, H. Yan, Angew. Chem. Int. Ed. 2006, 45, 730.
J. Simard, C. Briggs, A. K. Boal, V. M. Rotello, Chem. Commun. 2000, 1943.
J. Wang, L. Wang, X, Liu, Z. Liang, S. Song, W. Li, G. Li, C. Fan, Adv. Mater. 2007, 19, 3943.
Jayasena S D. (1999) Aptamers: an emerging class of molecules that rival antibodies in diagnostics. Clin Chem 45: 1628-1650.
Jenison, R. D., Gill, S. C., Pardi, A. & Polisky, B. (1994). High-resolution molecular discrimination by RNA. Science (Washington, D.C., United States) 263: 1425-9.
Jhaveri S, Kirby R, Conrad R, Maglott E, Bowser M, Kennedy R, Glick G, Ellington A. (2000) Designed signaling aptamers that transduce molecular recognition to changes in fluorescence intensity. J. Am. Chem. Soc. 122: 2469-2473.
Jhaveri S, Rajendran M, Ellington A D. (2000) In vitro selection of signaling aptamers. Nat Biotechnol 18: 1293-1297.
Jones L A, Prabel G B, Glennon J J, Copeland M F, Kavlock R J, 1993, J. Agric. Food Chem. 41: 735-741.
Joyce G F. (1994) In vitro evolution of nucleic acids. Curr Opin Struct Biol 4: 331-336.
Joyce, G. F. (1999). Reactions Catalyzed by RNA and DNA Enzymes. In The RNA World, vol. 37 (Gesteland, R. F., Cech, T. R. & Atkins, J. F., ed.), pp. 687-9, Cold Spring Harbor Laboratory Press, Cold Spring Harbor, N.Y.
K. C. Grabar, R. G. Freeman, M. B. Hommer, M. J. Natan, Anal. Chem. 1995, 67, 735.
Kato, T., Takemura, T., Yano, K., Ikebukuro, K. & Karube, I. (2000). In vitro selection of DNA aptamers which bind to cholic acid. Biochim. Biophys. Acta 1493: 12-8.
Kawakami, J., Imanaka, H., Yokota, Y. & Sugimoto, N. (2000). In vitro selection of aptamers that act with Zn2+. J. Inorg. Biochem. 82: 197-206.
Kester H Z., 1992, Distillation Design. McGraw-Hill, Inc., New York, N.Y.
Kiga D, Futamura Y, Sakamoto K, Yokoyama S. (1998) An RNA aptamer to the xanthine/guanine base with a distinctive mode of purine recognition. Nucleic Acids Res 26: 1755-1760.
Klussmann, S., Nolte, A., Bald, R., Erdmann, V. A. & Fuerste, J. P. (1996). Mirror-image RNA that binds D-adenosine. Nat. Biotechnol. 14: 1112-5.
Kobe K A, Stone J P, 1940, J. Phys. Chem. 446: 629-633.
Koizumi, M. & Breaker, R. R. (2000). Molecular Recognition of cAMP by an RNA Aptamer. Biochemistry 39: 8983-92.
Kroschwitz J I, Howe-Grant M, 1998, Encyclopedia of the chemical technology: supplement volume: aerogels to xylylene polymers. New York: Wiley.
L. Wang, X. Liu, X. Hu, S. Song, C. Fan, Chem. Comm. 2006, 3780.
Laromaine, A.; Koh, L.; Murugesan, M.; Ulijn, R. V.; Stevens, M. M., J. Am. Chem. Soc. 2007, 129, 4156-4157.
Lato, S. M., Boles, A. R. & Ellington, A. D. (1995). In vitro selection of RNA lectins: Using combinatorial chemistry to interpret ribozyme evolution. Chem. Biol. 2: 291-303.
Lauhon C T, Szostak J W. (1995) RNA aptamers that bind flavin and nicotinamide redox cofactors. J Am Chem Soc 117: 1246-1257.
Lazarova Z, Peeva L, 1994, 32: 75-82.
Lee, J.-S.; Han, M. S.; Mirkin, C. A., Angew. Chem., Int. Ed. 2007, SUST-050.
Leggett D C, Jenkins T F, Miyares P H, 1990, Anal. Chem. 62: 1355-1356.
Leinonen H, 1996, Corrosion 52: 337-346.
Li J, Lu Y. (2000) A highly sensitive and selective catalytic DNA biosensor for lead ions. J Am Chem Soc 122: 10466-10467.
Li J, Zheng W, Kwon A H, Lu Y. (2000) In vitro selection and characterization of a highly efficient Zn(II)-dependent RNA-cleaving deoxyribozyme. Nucleic Acids Res 28: 481-488.
Li Y, Breaker R R. (1999) Phosphorylating DNA with DNA. Proc Natl Acad Sci USA 96: 2746-2751.
Li Y, Sen D. (1996) A catalytic DNA for porphyrin metallation. Nat Struct Biol 3: 743-7.
Li, D.; Yan, Y.; Wieckowska, A.; Willner, I., Chem. Comm. 2007, 3544-3546.
Li, H. X.; Rothberg, L. J., J. Am. Chem. Soc. 2004, 126, 10958-10961.
Li, H.; Rothberg, L., Anal. Chem. 2005, 77, 6229-6233.
Li, H.; Rothberg, L., Proc. Natl. Acad. Sci. U.S.A. 2004, 101, 14036-14039.
Li, J., Zheng, W., Kwon, A. H. & Lu, Y. (2000). In vitro selection and characterization of a highly efficient Zn(II)-dependent RNA-cleaving deoxyribozyme. Nucleic Acids Res. 28: 481-8.
Li, J.; Lu, Y., J. Am. Chem. Soc. 2000, 122, 10466-10467.
Li, J.; Zheng, W.; Kwon, A. H.; Lu, Y., Nucleic Acids Res. 2000, 28, 481-488.
Likidis Z, Schügerl K, 1987, Biotechnology Letters 9: 229-232.
Lim, I. I.; Ip, W.; Crew, E.; Njoki, P. N.; Mott, D.; Zhong, C. J.; Pan, Y.; Zhou, S., Langmuir 2007, 23, 826-833.
Link S, Wang Z, El-Sayed M. (1999) Alloy formation of gold-silver particles and the dependence of the plasmon absorption on their compositions. J Phys Chem B 103: 3529-3533.
Liu, J.; Brown, A. K.; Meng, X.; Cropek, D. M.; Istok, J. D.; Watson, D. B.; Lu, Y., Proc. Natl. Acad. Sci. U.S.A. 2007, 104, 2056.
Liu, J.; Lu, Y., Anal. Chem. 2003, 75, 6666-6672.
Liu, J.; Lu, Y., Angew. Chem., Int. Ed. 2006, 45, 90-94.
Liu, J.; Lu, Y., J. Am. Chem. Soc. 2003, 125, 6642-6643.
Liu, J.; Lu, Y., J. Am. Chem. Soc. 2005, 127, 12677-12683.
Liu, J.; Lu, Y., Nature Protocols 2006, 1, 246-252.
Liu, J.; Mazumdar, D.; Lu, Y., Angew. Chem., Int. Ed. 2006, 45, 7955-7959.
Lohse P A, Szostak J W. (1996) Ribozyme-catalysed amino-acid transfer reactions. Nature 381: 442-444.
Long F A, McDevit W F, 1952, Chem. Rev. 51: 119-169.
Lorsch J R, Szostak J W. (1994) In vitro evolution of new ribozymes with polynucleotide kinase activity. Nature 371: 31-36.
Lorsch, J. R. & Szostak, J. W. (1994). In vitro selection of RNA aptamers specific for cyanocobalamin. Biochemistry 33: 973-82.
Lu X, Han P, Zhang Y, Wang Y, Shi J, 2000, Chem. Eng. J. 78: 165-171.
Lu Y, Liu J, SIMPLE CATALYTIC DNA BIOSENSORS FOR IONS BASED ON COLOR CHANGES, application Ser. No. 09/605,558, USA.
Lu Y. (2002) New transition-metal-dependent DNAzymes as efficient endonucleases and as selective metal biosensors. Chemistry 8: 4589-4596
M. N. Stojanovic, P. de Prada, D. W. Landry, J. Am. Chem. Soc. 2001, 123, 4928.
Majerfeld, I. & Yarus, M. (1994). An RNA pocket for an aliphatic hydrophobe. Nat. Struct. Biol. 1: 287-92.
Majerfeld, I. & Yarus, M. (1998). Isoleucine:RNA sites with associated coding sequences. Rna 4: 471-8.
Mannironi, C., Di Nardo, A., Fruscoloni, P. & Tocchini-Valentini, G. P. (1997). In vitro selection of dopamine RNA ligands. Biochemistry 36: 9726-34.
Maoz R, Sagiv J. (1987) Penetration-controlled reactions in organized monolayer assemblies. 1. Aqueous permanganate interaction with monolayer and multilayer films of long-chain surfactants. Langmuir 3: 1034-1044.
Mateles R I, 1998, Penicillin: a paradigm for biotechnology. Chicago: Candida Corp.
Mirkin C A, Letsinger R L, Mucic R C, Storhoff J J. (1996) A DNA-based method for rationally assembling particles into macroscopic materials. Nature 382: 607-609.
Mirkin, C. A., Letsinger, L. R, Mucic, C. R, Storhoff, J. J, Elghanian R, (2002) Particles having polynucleotides attached thereto and uses therefor. U.S. Pat. No. 6,361,944 USA.
Mlakar, M.; Branica, M., Anal. Chim. Acta 1989, 221, 279-287.
Morrison and Boyd. 1973. Organic Chemistry. Boston
Mucic R, Herrlein M, Mirkin, C. A., Letsinger R. (1996) Synthesis and characterization of DNA with ferrocenyl groups attached to their 5′-termini: Electrochemical characterization of a redox-active nucleotide monolayer. Chem. Commun. 555.
N. C. Seeman, Nature 2003, 421, 427.
N. K. Navani, Y. Li, Curr. Opin. Chem. Biol. 2006, 10, 272.
Nishihama N, Hirai T, Komasawa I, 2001, Ind. Eng. Chem. Res. 40: 3085-3091.
Nissen, P., Hansen, J., Ban, N., Moore, P. B. & Steitz, T. A. (2000). The structural basis of ribosome activity in peptide bond synthesis. Science (Washington, D. C.) 289: 920-30.
Nolan, E. M.; Lippard, S. J., J. Am. Chem. Soc. 2007, 129, 5910.
Nolte, A., Klussmann, S., Bald, R., Erdmann, V. A. & Fuerste, J. P. (1996). Mirror-design of L-oligonucleotide ligands binding to L-arginine. Nature Biotechnology 14: 1116-9.
Nuzzo R, Fusco F, Allara D. (1987) Spontaneously organized molecular assemblies, 3. Preparation and properties of solution adsorbed monolayers of organic disulfides on gold surfaces. J Am Chem Soc 109: 2358.
Okazawa, A., Maeda, H., Fukusaki, E., Katakura, Y. & Kobayashi, A. (2000). In vitro selection of hematoporphyrin binding DNA aptamers. Bioorg. Med. Chem. Lett. 10: 2653-6.
P. Chen, C. He, J. Am. Chem. Soc. 2004, 126, 728.
Pan, T. & Uhlenbeck, O. C. (1992). A small metalloribozyme with a two-step mechanism. Nature 358: 560-3.
Pavlov, V.; Xiao, Y.; Shlyahovsky, B.; Miner, I., J. Am. Chem. Soc. 2004, 126, 11768-11769.
Piccirilli J A, McConnell T S, Zaug A J, Noller H F, Cech T R. (1992) Aminoacyl esterase activity of the Tetrahymena ribozyme. Science 256: 1420-1424.
Pierotti R A, 1976, Chem. Rev. 76: 717-726.
Preventing lead poisoning in young children, 4th revision; U.S. Department of Health and Human Services, Centers for Disease Control: Atlanta, Ga., 1991
Prudent J R, Uno T, Schultz P G. (1994) Expanding the scope of RNA catalysis. Science 264: 1924-1927.
Public Law 102-550; Residential Lead-Based Paint Hazard Reduction Act of the housing and Community Development Act of 1992; Pittsburgh, 1992, pp. 102-550.
Qiang Z, Adams C, 2004, Water Research 38: 2874-2890.
R. Nutiu, Y. Li, J. Am. Chem. Soc. 2003, 125, 4771.
R. P. G. Cruz, J. B. Withers, Y. Li, Chem. Biol. 2004, 11, 57.
R. R. Breaker, Nature 2004, 432, 838.
Rakow N A, Suslick K S. (2000) A colorimetric sensor array for odour visualization. Nature 406: 710-713.
Rakow, N. A.; Suslick, K. S., Nature 2000, 406, 710-713.
Rink, S. M., Shen, J.-C. & Loeb, L. A. (1998). Creation of RNA molecules that recognize the oxidative lesion 7,8-dihydro-8-hydroxy-2′-deoxyguanosine (8-oxodG) in DNA. Proc. Natl. Acad. Sci. U.S.A. 95: 11619-24.
Robertson M P, Ellington A D. (1999) In vitro selection of an allosteric ribozyme that transduces analytes to amplicons. Nat Biotechnol 17: 62-66.
Rohwer, H.; Rheeder, N.; Hosten, E., Anal. Chim. Acta 1997, 341, 263-268.
Roth A, Breaker R R. (1998) An amino acid as a cofactor for a catalytic polynucleotide. Proc Natl Acad Sci USA 95: 6027-6031.
Rusconi C P, Scardino E, Layzer J, Pitoc G A, Ortel T L, Monroe D, Sullenger B A. (2002) RNA aptamers as reversible antagonists of coagulation factor IXa. Nature 419: 90-94.
Rydberg J, Musikas C, Choppin G R, 1992, Principles and practices of solvent extraction. New York: M. Dekker.
S. D. Jhaveri, R. Kirby, R. Conrad, E. J. Maglott, M. Bowser, R. T. Kennedy, G. Glick, A. D. Ellington, J. Am. Chem. Soc. 2000, 122, 2469.
S. Si, T. K. Mandal, Langmuir, 2007, 23, 190.
S. W. Santoro, G. F. Joyce, Proc. Natl. Acad. Sci. U.S.A. 1997, 94, 4262.
Safavi, A.; Bagheri, M., Anal. Chim. Acta 2005, 530, 55-60.
Santoro S W, Joyce G F. (1997) A general purpose RNA-cleaving DNA enzyme. Proc Natl Acad Sci USA 94: 4262-4266.
Santoro, S. W.; Joyce, G. F.; Sakthivel, K.; Gramatikova, S.; Barbas, C. F., Ill, J. Am. Chem. Soc. 2000, 122, 2433-2439.
Santoro, S. W. & Joyce, G. F. (1998). Mechanism and utility of an RNA-cleaving DNA enzyme. Biochemistry 37: 13330-42.
Sassanfar, M. & Szostak, J. W. (1993). An RNA motif that binds ATP. Nature (London) 364: 550-3.
Sato, K.; Hosokawa, K.; Maeda, M., J. Am. Chem. Soc. 2003, 125, 8102-8103.
Schenk F J, Callery P, Cannett P M, Draft J R, Lehotay S J, 2002, J. AOAC Int. 85: 1177-1180.
Schügerl K, 1994, Solvent extraction in biotechnology: recovery of primary and secondary metabolites. Verlag Berlin Heidelberg: Speinger.
Seetharaman S, Zivarts M, Sudarsan N, Breaker R R. (2001) Immobilized RNA switches for the analysis of complex chemical and biological mixtures. Nat Biotechnol 19: 336-341.
Sen, D. & Geyer, C. R. (1998). DNA enzymes. Curr. Opin. Chem. Biol. 2: 680-7.
Sessler, J. L.; Melfi, P. J.; Seidel, D.; Gorden, A. E. V.; Ford, D. K.; Palmer, P. D.; Tait, C. D., Tetrahedron 2004, 60, 11089-11097.
Shafer-Peltier, K. E.; Haynes, C. L.; Glucksberg, M. R.; Van Duyne, R. P., J. Am. Chem. Soc. 2003, 125, 588-593.
Shaiu W L, Larson D D, Vesenka J, Henderson E. (1993) Atomic force microscopy of oriented linear DNA molecules labeled with 5 nm gold spheres. Nucleic Acids Res 21: 99-103.
Sigurdsson, S. T., Thomson, J. B. & Eckstein, F. (1998). Small ribozymes. Cold Spring Harbor Monogr. Ser. 35: 339-76.
Sillén L G, (1964) Stability constants of metal-ion complexes. Edition: 2d ed.
Singleton V L, 1961, Am. J. Enol. Vitic. 12: 1-8.
Siva Rama Rao C V, Venkateswara Rao K, Raviprasad A, Chiranjivi C., 1978, J. Chem. Eng. Data. 23: 23-25.
Smith J, Olson D, Armitage B. (1999) Molecular recognition of PNA-containing hybrids: Spontaneous assembly of helical cyanine dye aggregates on PNA templates. J. Am. Chem. Soc. 121: 2686-2695.
Soriaga M, Hubbard A. (1982) Determination of the orientation of aromatic molecules adsorbed on platinum electrodes: The influence of solute concentration. J Am Chem Soc 104.
Soukup G A, Breaker R R. (2000) Allosteric nucleic acid catalysts. Curr Opin Struct Biol 10: 318-325.
Stage-Zimmermann, T. K. & Uhlenbeck, O. C. (1998). Hammerhead ribozyme kinetics. RNA 4: 875-89.
Stojanovic M N, Landry D W. (2002) Aptamer-based colorimetric probe for cocaine. J Am Chem Soc 124: 9678-9679.
Storhoff J, Elghanian R, Mucic R, Mirkin C, Letsinger R L. (1998) One-pot colorimetric differentiation of polynucleotides with single base imperfections using gold particle probes. J Am Chem Soc 120: 1959-1964.
Storhoff, J. J.; Lazarides, A. A.; Mucic, R. C.; Mirkin, C. A.; Letsinger, R. L.; Schatz, G. C., J. Am. Chem. Soc. 2000, 122, 4640-4650.
Subba Rao D, Venkateswara Rao K, Ravi Prasad A, Chiranjivi C, 1979, Extraction of acetonitrile from aqueous solutions. 2. ternary liquid equilibria. J. Chem. Eng. Data. 24: 241-244.
Sun, L. Q., Cairns, M. J., Saravolac, E. G., Baker, A. & Gerlach, W. L. (2000). Catalytic nucleic acids: From lab to applications. Pharmacol. Rev. 52: 325-47.
Tabata M, Kumamoto M, Nishimoto J, 1994, Anal. Sci. 10: 383-388.
Tabata M, Kumamoto M, Nishimoto J, 1996, Anal. Chem. 68: 758-762.
Tang J, Breaker R R. (1997) Rational design of allosteric ribozymes. Chem Biol 4: 453-459.
Tang J, Breaker R R. (2000) Structural diversity of self-cleaving ribozymes. Proc Natl Acad Sci USA 97: 5784-5789.
Tanner, N. K. (1998). Biochemistry of hepatitis delta virus catalytic RNAs. Ribozymes Gene Ther. Cancer: 23-38.
Tao, J. & Frankel, A. D. (1996). Arginine-Binding RNAs Resembling TAR Identified by in Vitro Selection. Biochemistry 35: 2229-38.
Tarasow T M, Tarasow S L, Eaton B E. (1997) RNA-catalysed carbon-carbon bond formation. Nature 389: 54-57.
Thornton J D, 1992. Science and practice of liquid-liquid extraction (II): process chemistry and extraction operations in the hydrometallurgical, nuclear, pharmaceutical, and food industries. Oxford: Clarendon Press.
Timmons, Zisman. (1965) J. Phys. Chem. 69: 984-990.
Tompkins H, Allara D. (1974) The study of the gas-solid interaction of acetic acid with a cuprous oxide surface using reflection-absorption spectroscopy. J. Colloid and Interface Sci. 49410.
Travascio, P., Bennet, A. J., Wang, D. Y. & Sen, D. (1999). A ribozyme and a catalytic DNA with peroxidase activity: active sites versus cofactor-binding sites. Chemistry & Biology 6: 779-87.
Tsang J, Joyce G F. (1996) In vitro evolution of randomized ribozymes. Methods Enzymol 267: 410-426.
Tuerk C, Gold L. (1990) Systematic evolution of ligands by exponential enrichment: RNA ligands to bacteriophage T4 DNA polymerase. Science 249: 505-510.
U.S. Pat. No. 4,362,603.
U.S. Pat. No. 6,326,508.
U.S. Pat. No. 6,843,890.
U.S. Pat. No. 6,159,347.
Vaish N K, Heaton P A, Fedorova O, Eckstein F. (1998) In vitro selection of a purine nucleotide-specific hammerheadlike ribozyme. Proc Natl Acad Sci USA 95: 2158-2162.
Valadkhan, S. & Manley, J. L. (2001). Splicing-related catalysis by protein-free snRNAs. Nature (London, United Kingdom) 413: 701-7.
Van der Wal Sj., 1985, Chromatographia 20: 274-278.
Vianini, E., Palumbo, M. & Gatto, B. (2001). In vitro selection of DNA aptamers that bind L-tyrosinamide. Bioorganic & Medicinal Chemistry 9: 2543-8.
W. H. Yang, G. C. Schatz, R. P. Vanduyne, J. Chem. Phys. 1995, 103, 869.
W. Zhao, W. Chiuman, M. A. Brook, Y. Li, ChemBioChem 2007, 8, 727.
W. Zhao, Y. Gao, S. A. Kandadai, M. A. Brook, Y. Li, Angew. Chem. Int. Ed. 2006, 45, 2409.
Wallace S T, Schroeder R. (1998) In vitro selection and characterization of streptomycin-binding RNAs: recognition discrimination between antibiotics. RNA 4: 112-123.
Wallis M G, Streicher B, Wank H, von Ahsen U, Clodi E, Wallace S T, Famulok M, Schroeder R. (1997) In vitro selection of a viomycin-binding RNA pseudoknot. Chem Biol 4: 357-366.
Wallis, M. G., Von Ahsen, U., Schroeder, R. & Famulok, M. (1995). A novel RNA motif for neomycin recognition. Chem. Biol. 2: 543-52.
Walter, N. G. & Burke, J. M. (1998). The hairpin ribozyme: structure, assembly and catalysis. Curr. Opin. Chem. Biol. 2: 24-30.
Wang B., 2000, Investigation on emulsification and demulsification during penicillin extraction. Doctoral dissertation. The Chinese Academy of Sciences.
Wang D Y, Lai B H, Sen D. (2002) A general strategy for effector-mediated control of RNA-cleaving ribozymes and DNA enzymes. J Mol Biol 318: 33-43.
Wang, L.; Liu, X.; Hu, X.; Song, S.; Fan, C., Chem. Comm. 2006, 3780-3782.
Wang, Y., Killian, J., Hamasaki, K. & Rando, R. R. (1996). RNA Molecules That Specifically and Stoichiometrically Bind Aminoglycoside Antibiotics with High Affinities. Biochemistry 35: 12338-46.
Wang, Z.; Lee, J. H.; Lu, Y., Adv. Mater. Accepted.
Warren K W, 1995, Reduction of corrosion through improvements in desalting. Benelux Refinery Symposium, Lanaken, Belgium.
Watson, J. D., Section Title: General Biochemistry 1968, 235.
Wecker M, Smith D, Gold L. (1996) In vitro selection of a novel catalytic RNA: characterization of a sulfur alkylation reaction and interaction with a small peptide. RNA 2: 982-994.
Wei, H.; Li, B.; Li, J.; Wang, E.; Dong, S., Chem. Comm. 2007, 36, 3735-3737.
Wernette, D. P.; Mead, C.; Bohn, P. W.; Lu, Y., Langmuir 2007, 23, 9513-9521.
Werstuck, G. & Green, M. R. (1998). Controlling gene expression in living cells through small molecule-RNA interactions. Science (Washington, D.C.) 282: 296-8.
Whitesides, (1995) Proceedings of the Robert A. Welch Foundation 39th Conference On Chemical Research Nanophase Chemistry., Houston, Tex.
Wiegand T W, Janssen R C, Eaton B E. (1997) Selection of RNA amide synthases. Chem Biol 4: 675-683.
Williams, K. P., Liu, X.-H., Schumacher, T. N. M., Lin, H. Y., Ausiello, D. A., Kim, P. S. & Bartel, D. P. (1997). Bioactive and nuclease-resistant L-DNA ligand of vasopressin. Proc. Natl. Acad. Sci. U.S.A. 94: 11285-90.
Willner, I.; Zayats, M., Angew. Chem., Int. Ed. 2007, 46, 6408-6418.
Wilson C, Szostak J W. (1995) In vitro evolution of a self-alkylating ribozyme. Nature 374: 777-782.
Wilson D S, Szostak J W. (1999) In vitro selection of functional nucleic acids. Annu Rev Biochem 68: 611-647.
Wilson, C. & Szostak, J. W. (1998). Isolation of a fluorophore-specific DNA aptamer with weak redox activity. Chem. Biol. 5: 609-17.
Wu Y G, Tabata M, Takamuku T, Yamaguchi A, Kawaguchi T, Chung N H, 2001, Fluid Phase Equilib. 192: 1-12.
Xiao, Y.; Lubin, A. A.; Heeger, A. J.; Plaxco, K. W., Angew. Chem., Int. Ed. 2005, 44, 5456.
Y. & Sen, D. (1996). A catalytic DNA for porphyrin metallation. Nat. Struct. Biol. 3: 743-7.
Y. Liu, C. Lin, H. Li, H. Yan, Angew. Chem. Int. Ed. 2005, 44, 4333.
Y. Lu, Chem. Eur. J. 2002, 8, 4588.
Yang, Q., Goldstein, I. J., Mei, H.-Y. & Engelke, D. R. (1998). DNA ligands that bind tightly and selectively to cellobiose. Proc. Natl. Acad. Sci. U.S.A. 95: 5462-7.
Z. Deng, Y. Tian, S.-H. Lee, A. E. Ribbe, C. Mao, Angew. Chem. Int. Ed. 2005, 44, 3582.
Zhang B, Cech T R. (1997) Peptide bond formation by in vitro selected ribozymes. Nature 390: 96-100.
Zhao, W.; Chiuman, W.; Brook, M. A.; Li, Y., Chembiochem 2007, 8, 727-731.
Zhao, W.; Chiuman, W.; Lam, J. C. F.; McManus, S. A.; Chen, W.; Cui, Y.; Pelton, R.; Brook, M. A.; Li, Y., J. Am. Chem. Soc. 130 (11), 3610-3618, 2008.
Zhao, W.; Gonzaga, F.; Li, Y.; Brook, M. A., Adv. Mater. 2007, 19, 1766-1771.
Zhou, P.; Gu, B., Environ. Sci. Technol. 2005, 39, 4435-4440.
Zillmann M, Limauro S E, Goodchild J. (1997) In vitro optimization of truncated stem-loop II variants of the hammerhead ribozyme for cleavage in low concentrations of magnesium under non-turnover conditions. RNA 3: 734-747.
Zimmerman, J. M. & Maher, L. J., Iii (2002). In vivo selection of spectinomycin-binding RNAs. Nucleic Acids Res. 30: 5425-35.

1. A sensor system kit for detecting an analyte, consisting essentially of:
(a) a nucleic acid enzyme, wherein the nucleic acid enzyme cleaves a substrate in the presence of the analyte;
(b) the substrate for the nucleic acid enzyme, comprising a polynucleotide;
(c) an aggregator; and
(d) particles.

2. The sensor system kit of claim 1, wherein the nucleic acid enzyme comprises DNA.

3. The sensor system kit of claim 1, wherein the polynucleotide of the substrate is DNA or RNA.

4. The sensor system kit of claim 1, wherein the polynucleotide comprises at least 5 nucleotides.

5. The sensor system kit of claim 1, wherein the particles comprise a material selected from the group consisting of metals, semiconductors, and mixtures thereof.

6. The sensor system kit of claim 1, wherein the particles comprise a material selected from the group consisting of silver, gold, and mixtures thereof.

7. The sensor system kit of claim 1, wherein the analyte is selected from the group consisting of Pb(II), UO₂(II), Hg(II), As(III), Fe(III), Zn(II), Cu(II), Co(II), nitrogen fertilizers, pesticides, dioxin, phenols, 2,4-dichlorophenoxyacetic acid, glucose, insulin, hCG-hormone, HIV, HIV proteins, anthrax, small pox, nerve gases, TNT, DNT, cocaine and antibiotics.

8. The sensor system kit of claim 1, wherein the aggregator is a salt selected from the group consisting of NaCl, KCl, LiCl, NaBr, KBr, LiBr, and mixtures thereof.

9. The sensor system kit of claim 1, further comprising a quencher.

10. The sensor system kit of claim 1, further comprising a pH modifier.

11-12. (canceled)

13. A sensor system kit for detecting an analyte, comprising:
(a) a nucleic acid enzyme, wherein the nucleic acid enzyme cleaves a substrate in the presence of the analyte;
(b) the substrate for the nucleic acid enzyme, comprising a polynucleotide;
(c) an aggregator; and
(d) particles,
wherein the particles are not attached to oligonucleotides that are hybridized to the substrate.

14. The sensor system kit of claim 13, wherein the nucleic acid enzyme comprises DNA.

15. The sensor system kit of claim 13, wherein the polynucleotide of the substrate is DNA or RNA.

16. The sensor system kit of claim 13, wherein the polynucleotide comprises at least 5 nucleotides.

17. The sensor system kit of claim 13, wherein the particles comprise a material selected from the group consisting of metals, semiconductors, and mixtures thereof.

18. The sensor system kit of claim 13, wherein the particles comprise a material selected from the group consisting of silver, gold, and mixtures thereof.

19. The sensor system kit of claim 13, wherein the analyte is selected from the group consisting of Pb(II), UO₂(II), Hg(II), As(III), Fe(III), Zn(II), Cu(II), Co(II), nitrogen fertilizers, pesticides, dioxin, phenols, 2,4-dichlorophenoxyacetic acid, glucose, insulin, hCG-hormone, HIV, HIV proteins, anthrax, small pox, nerve gases, TNT, DNT, cocaine and antibiotics.

20. The sensor system kit of claim 13, wherein the aggregator is selected from the group consisting of NaCl, KCl, LiCl, NaBr, KBr, LiBr, and mixtures thereof.

21. The sensor system kit of claim 13, further comprising a quencher.

22. The sensor system kit of claim 13, further comprising a pH modifier.

23-24. (canceled)

25. A method of detecting an analyte, comprising:
mixing a sample with a sensor to form a product; and
mixing the product with an indicator;
wherein the sensor comprises:
(a) a nucleic acid enzyme, wherein the nucleic acid enzyme cleaves a substrate in the presence of the analyte;
(b) the substrate for the nucleic acid enzyme, comprising a polynucleotide; and
(c) an aggregator, and
the indicator comprises:
(d) particles, wherein the particles aggregate in the presence of the
aggregator unless in the presence of sufficient ssDNA.

26. The method of claim 25, wherein the aggregator is an ionic strength modifier.

27. The method of claim 25, wherein the aggregator is selected from the group consisting of NaCl, KCl, LiCl, NaBr, KBr, LiBr, and mixtures thereof.

28. The method of claim 25, further comprising adding a quencher to the product.

29. The method of claim 25, further comprising adding a pH modifier to the product.

30. The method of claim 25, further comprising analyzing the indicator for a color change.

31. The method of claim 25, further comprising incubating the product for a maximum of 6 minutes prior to mixing the product with the indicator.

32. The method of claim 25, wherein the quantity of an analyte in the sample is inversely proportional to the formation or precipitation of aggregated particles.

33. The method of claim 25, where the sample comprises an analyte selected from the group consisting of nitrogen fertilizers, pesticides, dioxin, phenols, 2,4-dichlorophenoxyacetic acid, Pb(II), UO₂(II), Hg(II), As(III), Fe(III), Zn(II), Cu(II), Co(II), glucose, insulin, hCG-hormone, HIV, HIV proteins, anthrax, small pox, nerve gases, TNT, DNT, cocaine and antibiotics.

34. The method of claim 25, wherein the sample comprises a biological sample.

35. The method of claim 25, wherein the nucleic acid enzyme comprises DNA.

36-37. (canceled)

38. The method of claim 27, wherein the concentration of the aggregator is at least 100 mM.

39. An analytical test for an analyte, comprising:
(a) a base, having a reaction area and a visualization area,
(b) a capture species, on the base in the visualization area, and
(c) analysis chemistry reagents, on the base in0 the reaction area, consisting essentially of
(i) a nucleic acid enzyme,
(ii) a substrate, and
(iii) particles
wherein the analysis chemistry reagents can react with a sample comprising the analyte and water, to produce a product comprising nucleic acid, and
the capture species can bind the particles.

40. The sensor system kit of claim 39, wherein the nucleic acid enzyme comprises DNA.

41. The sensor system kit of claim 39, wherein the polynucleotide of the substrate is DNA or RNA.

42. The sensor system kit of claim 39, wherein the polynucleotide comprises at least 5 nucleotides.

43. The sensor system kit of claim 39, wherein the particles comprise a material selected from the group consisting of metals, semiconductors, and mixtures thereof.

44. The sensor system kit of claim 39, wherein the particles comprise a material selected from the group consisting of silver, gold, and mixtures thereof.

45. An analytical test for an analyte, comprising:
(a) a base, having a reaction area and a visualization area,
(b) a capture species, on the base in the visualization area, and
(c) analysis chemistry reagents, on the base in the reaction area, comprising
(i) a nucleic acid enzyme,
(ii) a substrate, and
(iii) particles
wherein the analysis chemistry reagents can react with a sample comprising the analyte and water, to produce a product comprising nucleic acid,
the particles are not attached to oligonucleotides that are hybridized to the substrate, and
the capture species can bind the particles.

46. The sensor system kit of claim 45, wherein the nucleic acid enzyme comprises DNA.

47. The sensor system kit of claim 45, wherein the polynucleotide of the substrate is DNA or RNA.

48. The sensor system kit of claim 45, wherein the polynucleotide comprises at least 5 nucleotides.

49. The sensor system kit of claim 45, wherein the particles comprise a material selected from the group consisting of metals, semiconductors, and mixtures thereof.

50. The sensor system kit of claim 45, wherein the particles comprise a material selected from the group consisting of silver, gold, and mixtures thereof.

51. An analytical test for an analyte, comprising:
(a) a base, having a reaction area and a visualization area,
(b) particles, on the base in the visualization area, and
(c) analysis chemistry reagents, on the base in the reaction area, comprising
(i) a nucleic acid enzyme,
(ii) a substrate,
wherein the analysis chemistry reagents can react with a sample comprising the analyte and water, to produce a visualization species comprising nucleic acid.

52. The sensor system kit of claim 51, wherein the nucleic acid enzyme comprises DNA.

53. The sensor system kit of claim 51, wherein the polynucleotide of the substrate is DNA or RNA.

54. The sensor system kit of claim 51, wherein the polynucleotide comprises at least 5 nucleotides.

55. The sensor system kit of claim 51, wherein the particles comprise a material selected from the group consisting of metals, semiconductors, and mixtures thereof.

56. The sensor system kit of claim 51, wherein the particles comprise a material selected from the group consisting of silver, gold, and mixtures thereof.
